(19) United States
(12) Patent Application Publication
(10) Pub. No.: US 2025/0273297 A1
(43) Pub. Date: Aug. 28, 2025

(54) METHODS AND DEVICES FOR PREDICTING DIMERIZATION IN NUCLEIC ACID AMPLIFICATION REACTION

(71) Applicant: SEEGENE, INC., Seoul (KR)

(72) Inventors: Jong Ha JANG, Seoul (KR); Hyun Ho KIM, Seoul (KR); Bo Kyu SHIN, Seoul (KR); Dong Min JUNG, Goyang-si (KR)

(21) Appl. No.: 19/094,440

(22) Filed: Mar. 28, 2025

Related U.S. Application Data
(63) Continuation of application No. PCT/KR2023/015137, filed on Sep. 27, 2023.

(30) Foreign Application Priority Data
Sep. 30, 2022 (KR) 10-2022-0125305

(51) Int. Cl. G16B 30/00; G16B 40/20

(52) U.S. Cl. CPC G16B 30/00; G16B 40/20

(57) ABSTRACT

Proposed is a computer-implemented method for predicting a dimerization in a nucleic acid amplification reaction. The method may include accessing a dimer prediction model learned by a transfer learning method, and providing an input data to the dimer prediction model. The input data may include a sequence data of an oligonucleotide. The method may also include obtaining a prediction result for the dimerization of the oligonucleotide from the dimer prediction model.

### CROSS REFERENCE TO RELATED APPLICATIONS

This is a continuation application of International Patent Application No. PCT/KR2023/015137, filed on Sep. 27, 2023, which claims priority to Korean patent application No. 10-2022-0125305 filed on Sep. 30, 2022, contents of each of which are incorporated herein by reference in their entireties.

### BACKGROUND

### Technical Field

The present disclosure relates to a computer-implemented method for predicting a dimerization in a nucleic acid amplification reaction, a computer-implemented method for obtaining a model for predicting a dimerization, and a computer device for performing the same.

### Description of Related Technology

Molecular diagnostic technologies are currently growing rapidly in the in vitro diagnostic market for early diagnosis of disease. Among them, methods using nucleic acids are usefully used for diagnosing causative genetic factors caused by infection by viruses and bacteria based on high specificity and sensitivity.

### SUMMARY

One aspect is a method for efficiently and accurately predicting a dimerization of oligonucleotides used for amplifying and detecting a plurality of target nucleic acid molecules.

Another aspect is a method capable of securing sufficient prediction accuracy even when using a small amount of labeled training data.

Another aspect is a method for accurately predicting a dimerization in consideration of various reaction conditions.

Another aspect is a method for accurately predicting a dimerization of a plurality of oligonucleotides even in a multiplex environment.

However, aspects of the present disclosure are not limited to the foregoing, and other unmentioned objects would be apparent to one of ordinary skill in the art from the following description.

Another aspect is a computer-implemented method for predicting a dimerization in a nucleic acid amplification reaction, comprising: accessing a dimer prediction model learned by a transfer learning method; providing an input data to the dimer prediction model, wherein the input data comprise a sequence data of an oligonucleotide; and obtaining a prediction result for the dimerization of the oligonucleotide from the dimer prediction model.

In an embodiment, wherein the oligonucleotide comprises a primer.

In an embodiment, wherein the oligonucleotide comprises a forward primer and a reverse primer.

In an embodiment, wherein the dimerization comprises at least one selected from the group consisting of (i) a dimerization formed between two or more oligonucleotides and (ii) a dimerization formed in one oligonucleotide.

In an embodiment, wherein the dimer prediction model is a model obtained by fine-tuning a pre-trained model.

In an embodiment, wherein the pre-trained model uses a plurality of nucleic acid sequences as a training data.

In an embodiment, wherein the plurality of nucleic acid sequences are obtained from a specific group of an organism.

In an embodiment, wherein the pre-trained model is trained by a semi-supervised learning method performed in which a mask to some of bases in the nucleic acid sequences is applied and then an answer of the masked base is found.

In an embodiment, wherein the pre-trained model is trained by using nucleic acid sequences tokenized with tokens each having two or more bases.

In an embodiment, wherein the tokens comprise each bases tokenized by (i) dividing the nucleic acid sequences by k unit (wherein k is a natural number) or (ii) dividing the nucleic acid sequences by a function unit.

In an embodiment, wherein the fine-tuning is performed using a plurality of training data sets, each training data set comprises (i) a training input data comprising a sequence data of two or more oligonucleotides and (ii) a training answer data comprising a label data as to occurrence and/or non-occurrence of dimer of the two or more oligonucleotides.

In an embodiment, wherein the fine-tuning comprises (i) joining sequences of the two or more oligonucleotides by using a discrimination token and (ii) tokenizing the joined sequences to obtain a plurality of tokens.

In an embodiment, wherein the fine-tuning comprises (i) predicting a dimerization of the two or more oligonucleotides using a context vector generated from the plurality of tokens, and (ii) training the pre-trained model for reducing a difference between the predicted result and the training answer data.

In an embodiment, wherein the dimer prediction model comprises a plurality of models generated by fine-tuning the pre-trained model in accordance with each of reaction conditions used in the nucleic acid amplification reaction.

In an embodiment, wherein the obtaining the prediction result comprises obtaining a plurality of prediction results for the dimerization from the plurality of models or obtaining a prediction result from a model corresponding to a reaction condition matched to the input data among the plurality of models.

In an embodiment, wherein the training input data further comprises a data of a reaction condition used in the nucleic acid amplification reaction, and the dimer prediction model comprises one model generated by fine-tuning the pre-trained model using the plurality of training data sets.

In an embodiment, wherein the input data further comprises a data of a reaction condition, whereby the prediction result for the dimerization is obtained based on the sequence data and the data of the reaction condition.

In an embodiment, wherein the reaction condition is a reaction medium, a reaction temperature and/or a reaction time used in the nucleic acid amplification reaction.

In an embodiment, wherein the reaction medium comprises at least one selected from the group consisting of a pH-related material, an ion strength-related material, an enzyme, and an enzyme stabilization-related material.

In an embodiment, wherein the pH-related material comprises a buffer, the ion strength-related material comprises an ionic material, and the enzyme stabilization-related material comprises a sugar.

In an embodiment, wherein the computer-implemented method further comprises outputting a prediction supporting data used as a basis for prediction of the dimerization.

In an embodiment, wherein the prediction supporting data is calculated by XAI (explainable artificial intelligence) method.

In an embodiment, wherein the computer-implemented method further comprises providing a predicted image representing the dimerization.

Another aspect is a computer program stored on a computer-readable recording medium, the computer program including instructions that, when executed by one or more processors, enable the one or more processors to perform a method for predicting a dimerization in a nucleic acid amplification reaction by a computer device, the method comprising: accessing a dimer prediction model learned by a transfer learning method; providing an input data to the dimer prediction model, wherein the input data comprise a sequence data of an oligonucleotide; and obtaining a prediction result for the dimerization of the oligonucleotide from the dimer prediction model.

Another aspect is a computer-readable recording medium storing a computer program including instructions that, when executed by one or more processors, enable the one or more processors to perform a method for predicting a dimerization in a nucleic acid amplification reaction by a computer device, the method comprising: accessing a dimer prediction model learned by a transfer learning method; providing an input data to the dimer prediction model, wherein the input data comprise a sequence data of an oligonucleotide; and obtaining a prediction result for the dimerization of the oligonucleotide from the dimer prediction model.

Another aspect is a computer device for predicting a dimerization in a nucleic acid amplification reaction, the computer device comprising: a processor; and a memory that stores one or more instructions that, when executed by the processor, cause the computer device to perform operations, the operations comprising: accessing a dimer prediction model learned by a transfer learning method; providing an input data to the dimer prediction model, wherein the input data comprise a sequence data of an oligonucleotide; and obtaining a prediction result for the dimerization of the oligonucleotide from the dimer prediction model.

Another aspect is a computer-implemented method for predicting a dimerization in a nucleic acid amplification reaction, comprising: accessing a dimer prediction model obtained by fine-tuning a pre-trained model, providing an input data to the dimer prediction model, wherein the input data comprise a sequence data of an oligonucleotide; and obtaining a prediction result for the dimerization of the oligonucleotide from the dimer prediction model, wherein the pre-trained model uses a plurality of nucleic acid sequences as a training data, wherein the pre-trained model is trained by a semi-supervised learning method performed in which nucleic acid sequences are tokenized with tokens each having two or more bases, a mask to some of bases in the nucleic acid sequences is applied, and then an answer of the masked base is found.

In an embodiment, wherein the fine-tuning is performed using a plurality of training data sets, each training data set comprises (i) a training input data comprising a sequence data of two or more oligonucleotides and (ii) a training answer data comprising a label data as to occurrence and/or non-occurrence of dimer of the two or more oligonucleotides.

In an embodiment, wherein the fine-tuning comprises (i) joining sequences of the two or more oligonucleotides by using a discrimination token, (ii) tokenizing the joined sequences to obtain a plurality of tokens, (iii) predicting a dimerization of the two or more oligonucleotides using a context vector generated from the plurality of tokens, and (iv) training the pre-trained model for reducing a difference between the predicted result and the training answer data.

Another aspect is a computer-implemented method for obtaining a dimer prediction model for predicting a dimerization in a nucleic acid amplification reaction comprising: obtaining a pre-trained model; and obtaining the dimer prediction model by fine-tuning the pre-trained model, wherein the fine-tuning is performed using a plurality of training data sets, each training data set comprises (i) a training input data comprising a sequence data of two or more oligonucleotides and (ii) a training answer data comprising a label data as to occurrence and/or non-occurrence of dimer of the two or more oligonucleotides.

In an embodiment, wherein the pre-trained model uses a plurality of nucleic acid sequences as a training data.

In an embodiment, wherein the plurality of nucleic acid sequences are obtained from a specific group of an organism.

In an embodiment, wherein the obtaining the pre-trained model comprises training the pre-trained model by a semi-supervised learning method performed in which nucleic acid sequences are tokenized with tokens each having two or more bases, a mask to some of bases in the nucleic acid sequences is applied, and then an answer of the masked base is found.

In an embodiment, wherein the obtaining the dimer prediction model by fine-tuning the pre-trained model comprises (i) joining sequences of the two or more oligonucleotides by using a discrimination token, and (ii) tokenizing the joined sequences to obtain a plurality of tokens.

In an embodiment, wherein the obtaining the dimer prediction model by fine-tuning the pre-trained model further comprises (iii) predicting a dimerization of the two or more oligonucleotides using a context vector generated from the plurality of tokens, and (iv) training the pre-trained model for reducing a difference between the predicted result and the training answer data.

In an embodiment, wherein the obtaining the dimer prediction model by fine-tuning the pre-trained model comprises generating a plurality of dimer prediction models by fine-tuning the pre-trained model in accordance with each of reaction conditions used in the nucleic acid amplification reaction.

In an embodiment, wherein the training input data further comprises a data of a reaction condition used in the nucleic acid amplification reaction, and wherein the obtaining the dimer prediction model by fine-tuning the pre-trained model comprises generating one dimer prediction model by fine-tuning the pre-trained model using the plurality of training data sets.

In an embodiment, wherein the reaction condition is a reaction medium, a reaction temperature and/or a reaction time used in the nucleic acid amplification reaction.

In an embodiment, wherein the reaction medium comprises at least one selected from the group consisting of a pH-related material, an ion strength-related material, an enzyme, and an enzyme stabilization-related material.

According to one embodiment of the present disclosure, it is possible to efficiently and accurately predict a dimerization of oligonucleotides used for amplifying and detecting a plurality of target nucleic acid molecules.

In addition, as a dimer prediction model learned by the transfer learning method is used, sufficient prediction accuracy can be secured even when using a small amount of labeled training data.

Further, it is possible to accurately predict the dimerization of oligonucleotides in consideration of various reaction conditions. Also, it is possible to accurately predict the dimerization of a plurality of oligonucleotides even in a multiplex environment. Accordingly, the detection accuracy of the target nucleic acid molecule can be improved.

The effects of the present disclosure are not limited to the foregoing, and should be understood to include all effects that can be inferred from the configuration of the present disclosure described in the detailed description or claims of the present disclosure.

### BRIEF DESCRIPTION OF THE DRAWINGS

FIG. 1 is a block diagram illustrating a computer device according to an embodiment.

FIG. 2 is a view illustrating a concept of pre-training process according to an embodiment.

FIG. 3 is a view illustrating a structure and an operation of a BERT-based language model in the pre-training process according to an embodiment.

FIG. 4 is a view illustrating an example method for predicting probabilities per base by the pre-trained model in the computer device according to an embodiment.

FIG. 5 is a view illustrating a concept of fine-tuning process according to an embodiment.

FIG. 6 is a view illustrating a concept of reaction conditions according to an embodiment.

FIG. 7 is a view illustrating a structure and an operation of a BERT-based dimer prediction model in the fine-tuning process according to an embodiment.

FIG. 8 is an exemplary flowchart for obtaining a dimer prediction model according to an embodiment.

FIG. 9 is a view illustrating a concept of an inference operation by the dimer prediction model according to an embodiment.

FIG. 10 is a view illustrating an example method for predicting a dimerization probability by the dimer prediction model according to an embodiment.

FIG. 11 is a view illustrating an example process in which a sequence data of an oligonucleotide is obtained through a user input according to an embodiment.

FIG. 12 is a view illustrating an example process in which a prediction result for the dimerization is output according to a first embodiment.

FIG. 13 is a view illustrating an example process in which the dimer prediction is performed using a plurality of oligonucleotide sequence sets of according to an embodiment.

FIG. 14 is a view illustrating an example process in which the computer device provides a predicted image representing the dimerization according to an embodiment.

FIG. 15 is an exemplary flowchart for predicting a dimerization in a nucleic acid amplification reaction by the computer device according to an embodiment.

FIG. 16 is a schematic diagram illustrating a computing environment according to an exemplary embodiment of the present disclosure.

### DETAILED DESCRIPTION

Most of the diagnostic methods using nucleic acids include methods using nucleic acid amplification reaction amplifying target nucleic acid (e.g., viral or bacterial nucleic acid). As a representative example, polymerase chain reaction (PCR) among nucleic acid amplification reactions includes repeated cycles of denaturation process of double-stranded DNA, annealing process of oligonucleotide primers to a DNA template, and extension process of primers by DNA polymerase (Mullis et al., U.S. Pat. Nos. 4,683,195, 4,683,202 and 4,800,159; Saiki et al., Science 230:1350-1354 (1985)).

PCR-based technologies are widely used in scientific applications or methods in biological and medical research fields as well as amplification of target DNA sequences, such as reverse transcription PCR (RT-PCR), differential display PCR (DD-PCR), cloning of known or unknown genes by PCR, rapid amplification of cDNA ends (RACE), arbitrarily primed PCR (AP-PCR), multiplex PCR, SNP genome typing, and PCR-based genomic analysis (McPherson and Moller, (2000) PCR. BIOS Scientific Publishers, Springer-Verlag New York Berlin Heidelberg, NY).

Other methods for amplifying nucleic acids include following methods: Ligase Chain Reaction (LCR), Strand Displacement Amplification (SDA), Nucleic Acid Sequence-Based Amplification (NASBA), Transcription Mediated Amplification (TMA), Recombinase Polymerase Amplification (RPA), Loop-mediated isothermal amplification (LAMP), and Rolling Amplification (RCA)-Circle Amplification).

Recently, multiplex diagnostic technologies for detecting a plurality of target nucleic acids in one tube based on such a nucleic acid amplification reaction is used. For example, there are various multiplex technologies for detecting various types of viruses at once using methods such as PCR and LAMP etc. described above as examples of nucleic acid amplification reactions.

As a representative example, multiplex PCR among PCR-based technologies means a technology amplifying and detecting a plurality of regions in a plurality of target nucleic acid molecules simultaneously by using a combination of a plurality of oligonucleotide sets (e.g., forward and reverse primers, and probes) in one tube.

For providing a combination of a plurality of oligonucleotide sets for multiplex PCR, an oligonucleotide set having performance capable of detecting a plurality of nucleic acid sequences in a specific target nucleic acid molecule with maximum coverage should be designed. Also, a pool of oligonucleotide sets comprising such oligonucleotide sets should be provided. The oligonucleotides (e.g., primers and probes) comprised in the oligonucleotide set are designed in consideration of the Tm value and the length of nucleotides, and the oligonucleotide set is provided in consideration of the amplicon size and dimer formation.

For providing performing the multiplex PCR using such oligonucleotide sets, it is important that there is no interference between a plurality of oligonucleotide sets. Dimerization is one of the representative phenomena of such interference. Even if the characteristics of the oligonucleotide set are excellent, when a dimer is formed between oligonucleotide sets designed for detecting different target nucleic acid molecules, the combinations of oligonucleotide sets cannot be provided because the possibility of failing to accurately detect the target nucleic acid molecule increases.

Accordingly, a dimer prediction technology for accurately predicting in advance whether a dimer will be formed between oligonucleotide sets is used.

As a conventional dimer prediction technology, a pattern rule-based prediction method for determining whether a dimer is formed by comparing sequences of oligonucleotide sets according to predetermined pattern rules is used. However, since this conventional technology has a limitation in not considering the diversity of the experimental environment because the prediction is made based only on the predetermined pattern rules for oligonucleotides. In addition, there is a problem in that prediction efficiency is low because it is difficult to develop a new pattern rule related to dimerization.

In addition, when the size of the pool of oligonucleotide sets is large or the number of oligonucleotides included in the oligonucleotide set is large, it takes a long time to determine whether dimers are formed in various candidate combinations. Further, it is difficult to determine dimerization whether dimers are formed in all candidate combinations. Accordingly, there is a problem in that dimer prediction ability decreases.

Various exemplary embodiments are described with reference to the drawings. In the present specification, various descriptions are presented for understanding the present disclosure. Prior to describing the specific content for carrying out the present disclosure, it should be noted that the configurations that are not directly related to the technical gist of the present disclosure are omitted within the scope of not disturbing the technical gist of the present disclosure. Further, the terms or words used in the present specification and the claims should be interpreted to have meanings and concepts consistent with the technical spirit of the present disclosure based on the principle that the inventor can define the concept of an appropriate term to best describe the invention.

Terms such as “component”, “module”, “system”, “unit”, and the like used in the present specification indicate a computer-related entity, hardware, firmware, software, a combination of software and hardware, or execution of software. For example, a component may be a processing step which is executed in a processor, a processor, an object, an execution thread, a program and/or a computer, but is not limited thereto. For example, both an application which is executed in a computing device and a computing device may be a component. One or more components may be stayed within the processor and/or execution thread. One component may be localized in one computer. One component may be distributed between two or more computers. Such components may be executed from various computer readable media having various data structures stored therein. The components may communicate with each other through local and/or remote processings in accordance with a signal (for example, data transmitted through other system and a network such as Internet through data and/or a signal from one component which interacts with other component in a local system or a distributed system) having one or more data packets.

A term “or” is intended to refer to not exclusive “or”, but inclusive “or”. That is, when it is not specified or unclear on the context, “X uses A or B” is intended to mean one of natural inclusive substitutions. That is, when X uses A; X uses B; or X uses both A and B, “X uses A or B” may be applied to any of the above instances. Further, it should be understood that the term “and/or” used in this specification designates and includes all available combinations of one or more items among listed related items.

A term “comprise” and/or “comprising” is understood that the corresponding feature and/or component are present. However, it should be understood that a term “include” and/or “including” does not preclude existence or addition of one or more other features, constituent elements and/or a group of them. Further, when it is not separately specified or it is not clear from the context to indicate a singular form, the singular form in the specification and the claims is generally interpreted to represent “one or more”.

A term “at least one of A or B” of “at least one of A and B” should be interpreted to mean “the case including only A”, “the case including only B”, and “the case where A and B are combined”.

Those skilled in the art will further appreciate that the various illustrative logical blocks, configurations, modules, circuits, means, logics, and algorithm steps described in connection with the embodiments disclosed herein may be implemented as electronic hardware, computer software, or combinations thereof. To clearly illustrate the interchangeability of hardware and software, various illustrative components, blocks, configurations, means, logics, modules, circuits, and steps have been described above generally in terms of their functionality. Whether the functionality is implemented as hardware or software depends on a specific application and design constraints imposed on the overall system. Those skilled in the art may implement the described functionality in various ways for each of specific applications. However, decisions of such implementations should be interpreted without departing from the scope of the present disclosure.

Description of the suggested exemplary embodiments is provided to allow those skilled in the art to use or embody the present disclosure. Various modifications to these embodiments may be apparent to those skilled in the art. Generic principles defined herein may be applied to other embodiments without departing from the scope of the present disclosure. Therefore, the present disclosure is not limited to the embodiments suggested herein. The present disclosure needs to be interpreted within the broadest scope consistent with principles suggested herein and novel features.

In the present disclosure, terms expressed as an Nth, such as a first, a second, or a third, are used to distinguish at least one entity. For example, the entities represented by a first and a second may be the same as or different from each other.

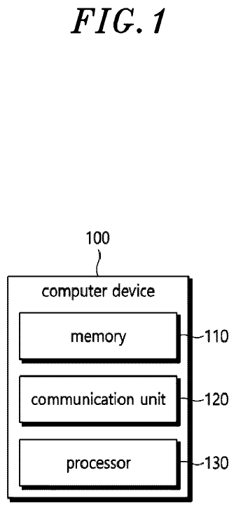

Prior to describing FIG. 1, terms used herein are described.

In the disclosure, the term “oligonucleotide” refers to a linear oligomer of natural or modified monomers or linkages. The oligonucleotide includes deoxyribonucleotides and ribonucleotides, can specifically hybridize with a target nucleotide sequence, and is naturally present or artificially synthesized. An oligonucleotide is especially a single chain for hybridization. maximal efficiency in Specifically, the oligonucleotide is an oligodeoxyribonucleotide. The oligonucleotide of the present invention may include naturally occurring dNMPs (i.e., dAMP, dGMP, dCMP and dTMP), nucleotide analogs, or derivatives. The oligonucleotide may also include a ribonucleotide. For example, the oligonucleotide used in the present invention may include nucleotides with backbone modifications, such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides with base modifications, such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine. Especially, the term “oligonucleotide” used herein is a single strand composed of a deoxyribonucleotide. The term “oligonucleotide” includes oligonucleotides that hybridize with cleavage fragments which occur depending on a target nucleic acid sequence. Especially, the oligonucleotide includes a primer and/or a probe.

In the disclosure, the term “primer” refers to an oligonucleotide that can act as a point of initiation of synthesis under conditions in which synthesis of primer extension products complementary to a target nucleic acid strand (a template) is induced, i.e., in the presence of nucleotides and a polymerase, such as DNA polymerase, and under appropriate temperature and pH conditions. The primer needs to be long enough to prime the synthesis of extension products in the presence of a polymerase. An appropriate length of the primer is determined according to a plurality of factors, including temperatures, fields of application, and primer sources.

The length of the primer is, for example, 10 to 100 nucleotides, 10 to 80 nucleotides, 10 to 50 nucleotides, 10 to 40 nucleotides, 10 to 30 nucleotides, 15 to 100 nucleotides, 15 to 80 nucleotides, 15 to 50 nucleotides, 15 to 40 nucleotides, 15 to 30 nucleotides, 20 to 100 nucleotides, 20 to 80 nucleotides, 20 to 50 nucleotides, 20 to 40 nucleotides, or 20 to 30 nucleotides. In cases where the primer is a DPO primer developed by the present applicant (see U.S. Pat. No. 8,092,997), the descriptions for the length of the DPO primer disclosed in the patent document is incorporated herein by reference.

In the disclosure, the term “probe” refers to a single-stranded nucleic acid molecule containing a portion or portions that are complementary to a target nucleic acid sequence. The probe may also contain a label capable of generating a signal for target detection. The term “probe” can refer to an oligonucleotide or a group of oligonucleotides which is involved in providing a signal indicating the presence of a target nucleic acid sequence.

The length of the probe is, for example, 10 to 100 nucleotides, 10 to 80 nucleotides, 10 to 50 nucleotides, 10 to 40 nucleotides, 10 to 30 nucleotides, 15 to 100 nucleotides, 15 to 80 nucleotides, 15 to 50 nucleotides, 15 to 40 nucleotides, 15 to 30 nucleotides, 20 to 100 nucleotides, 20 to 80 nucleotides, 20 to 50 nucleotides, 20 to 40 nucleotides, or 20 to 30 nucleotides. In cases where the probe is a tagging probe, the description of the length applies to the targeting region of the tagging probe. The length of the tagging site of the tagging probe is not particularly limited, for example, 7 to 48 nucleotides, 7 to 40 nucleotides, 7 to 30 nucleotides, 7 to 20 nucleotides, 10 to 48 nucleotides, 10 to 40 nucleotides, 10 to 30 nucleotides, 10 to 20 nucleotides, 12 to 48 nucleotides, 12 to 40 nucleotides, 12 to 30 nucleotides, or 12 to 20 nucleotides.

The oligonucleotides may include a typical primer and probe composed of a sequence hybridizing with a target nucleic acid sequence. The oligonucleotide may be a primer and/or a probe used in various methods, comprising Scorpion method (Whitcombe etc., Nature Biotechnology 17:804-807(1999)), Sunrise (or Amplifluor) method (Nazarenko etc., Nucleic Acids Research, 25(12):2516-2521(1997), and U.S. Pat. Nos. 6,117,635), Lux method (U.S. Pat. Nos. 7,537,886), Plexor method (Sherrill CB, etc., Journal of the American Chemical Society, 126:4550-4556(2004)), Molecular beacon method (Tyagi etc., Nature Biotechnology v. 14 Mar. 1996), Hybeacon method (French DJ etc., Mol. Cell Probes, 15(6):363-374(2001)), adjacent hybridization probe method (Bernard P. S. etc., Anal. Biochem., 273:221(1999)), LNA method (U.S. Pat. Nos. 6,977,295), DPO method (WO 2006/095981), and PTO method (WO 2012/096523).

The oligonucleotide refers to one or more oligonucleotides. In an embodiment, the term “oligonucleotide” may be interpreted as a concept including a sequence set of oligonucleotides paired with a forward sequence and a reverse sequence. In an embodiment, the oligonucleotide may include a primer set of a forward primer and a reverse primer. For example, the forward primer is a primer annealing with an antisense strand, a non-coding strand, or a template strand. Also, the forward primer may be a primer that can act as a point of initiation of a coding or positive strand of a target analyte. In addition, the reverse primer is a primer annealing with a 3′ end of a sense strand or the coding strand. Also, the reverse primer may be a primer that can act as a point of initiation for synthesizing a complementary strand of the coding sequence or non-coding sequence of the target analyte.

Here, the above-mentioned forward primer and reverse primer may refer to a pair of primers determining a specific amplification region in a target nucleic acid sequence in an embodiment. The forward primer and reverse primer may refer to individual primers not operating as a pair in another embodiment.

In the disclosure, the term “target nucleic acid sequence” refers to a particular target nucleic acid sequence representing a target nucleic acid molecule. In addition, the nucleic acid sequence means that bases are arranged in order, wherein the base is one of the components of a nucleotide. For example, “nucleic acid sequence” can be used interchangeably herein with “base sequence”. Each of the individual bases constituting a nucleic acid sequence may correspond to one of four types of bases, for example, adenine (A), guanine (G), cytosine (C), and thymine (T).

In the disclosure, the term “analyte” may include a variety of substances (e.g., biological and non-biological substances), which may refer to the same target as the term “target analyte”. Specifically, the target analyte may include a biological substance, more specifically at least one of nucleic acid molecules (e.g., DNA and RNA), proteins, peptides, carbohydrates, lipids, amino acids, biological compounds, hormones, antibodies, antigens, metabolites, and cells.

In the disclosure, the term “target analyte” refers to a nucleotide molecule in any form of organism to be analyzed, obtained, or detected. The organism refers to an organism that belongs to one genus, species, subspecies, subtype, genotype, serotype, strain, isolate, or cultivar. In the disclosure, “organism” can be used interchangeably with “target analyte”.

Examples of the organism include prokaryotic cells (e.g., Mycoplasma pneumoniae, Chlamydophila pneumoniae, Legionella pneumophila, Haemophilus influenzae, Streptococcus pneumoniae, Bordetella pertussis, Bordetella parapertussis, Neisseria meningitidis, Listeria monocytogenes, Streptococcus agalactiae, Campylobacter, Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis, Mycoplasma genitalium, Ureaplasma urealyticum, Ureaplasma parvum, Mycobacterium tuberculosis, Treponema pallidum, Candida, Mobiluncus, Megasphaera, Lacto spp., Mycoplasma genitalium, Clostridium difficile, Helicobacter Pylori, ClariR, CPE, Group B Streptococcus, Enterobacter cloacae complex, Proteus mirabilis, Klebsiella aerogenes, Pseudomonas aeruginosa, Klebsiella oxytoca, Serratia marcescens, Klebsiella pneumoniae, Actinomycetaceae actinotignum, Enterococcus faecium Staphylococcus epidermidis, Enterococcus faecalis, Staphylococcus saprophyticus, Staphylococcus aureus, Acinetobacter baumannii, Morganella morganii, Aerococcus urinae, Pantoea aglomerans, Citrobacter Freundii, Providencia stuartii, Citrobacter koseri, Streptococcus anginosus, Trichophyton mentagrophytes complex, Microsporum spp., Trichophyton rubrum, Epidermophyton floccosum, Trichophyton tonsurans), eukaryotic cells (e.g., protozoa and parasites, fungi, yeasts, higher plants, lower animals, and higher animals including mammals and humans), viruses, or viroids. Examples of the parasites of the prokaryotic cells include Giardia lamblia, Entamoeba histolytica, Cryptosporidium, Blastocystis hominis, Dientamoeba fragilis, Cyclospora cayetanensis, stercoralis, trichiura, hymenolepis, Necator americanus, Enterobius vermicularis, Taenia spp., Ancylostoma duodenale, Ascaris lumbricoides, Enterocytozoon spp./Encephalitozoon spp. Examples of the viruses include: influenza A virus (Flu A), influenza B virus (Flu B), respiratory syncytial virus A (RSV A), respiratory syncytial virus B (RSV B), Covid-19 virus, parainfluenza virus 1 (PIV 1), parainfluenza virus 2 (PIV 2), parainfluenza virus 3 (PIV 3), parainfluenza virus 4 (PIV 4), metapneumovirus (MPV), Human enterovirus (HEV), human bocavirus (HBoV), human rhinovirus (HRV), coronavirus, and adenovirus, which cause respiratory diseases; and norovirus, rotavirus, adenovirus, astrovirus, and sapovirus, which cause gastrointestinal diseases. Examples of the viruses also include human papillomavirus (HPV), middle east respiratory syndrome-related coronavirus (MERS-CoV), dengue virus, herpes simplex virus (HSV), human herpes virus (HHV), Epstein-Barr virus (EMV), varicella zoster virus (VZV), cytomegalovirus (CMV), HIV, Parvovirus B19, Parechovirus, Mumps, Dengue virus, Chikungunya virus, Zika virus, West Nile virus, hepatitis virus, and poliovirus.

In an embodiment of the disclosure, the organism may be GBS serotype, Bacterial colony, or v600e. The organism in the present disclosure may include not only the virus described above but also various analysis targets such as bacteria and humans, and may be a specific region of a gene cut using CRISPR technology. The range of the organism is not limited to the above examples.

The above-described target analyte, particularly target nucleic acid molecules, may be amplified by various methods: polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991)), rolling circle amplification (RCA) (Lisby, Mol. Biotechnol. 12(1):75-99 (1999); Hatch et al., Genet. Anal. 15(2):35-40 (1999)) and Q-Beta Replicase) (Lizardi et al., BiolTechnology 6:1197(1988)), loop-mediated isothermal amplication (LAMP, Y. Mori, H. Kanda and T. Notomi, J. Infect. Chemother., 2013, 19, 404?411), recombinase polymerase amplication (RPA, J. Li, J. Macdonald and F. von Stetten, Analyst, 2018, 144, 31?67).

The amplification reaction for amplifying the signal indicating the presence of the target analyte may be performed in a manner in which the signal is also amplified while the target analyte is amplified (e.g., real-time PCR method). For example, the amplification reaction is carried out by PCR, specifically real-time PCR, or isothermal amplification reaction (e.g., LAMP or RPA). Alternatively, according to an embodiment, the amplification reaction may be performed in a manner in which only a signal indicating the presence of the target analyte is amplified without amplifying the target analyte (e.g., CPT method (Duck P, et al., Biotechniques, 9:142-148(1990)), Invader assay (U.S. Pat. Nos. 6,358,691 and 6,194,149)).

In the disclosure, the term “dimer” may mean a hybridization resultant of one or more oligonucleotides. Two regions substantially complementary to each other in one or more oligonucleotides may be hybridized with each other under a certain condition to form a hybridization resultant. Further, the term “dimerization” as used in the context of dimer may mean a phenomenon in which the two complementary regions in one or more oligonucleotides hybridize with each other to form a dimer.

In an embodiment, the dimerization may comprise at least one selected from the group consisting of (i) a dimerization formed between two or more oligonucleotides (e.g., pair-dimer) and (ii) a dimerization formed in one oligonucleotide (e.g., self-dimer).

The dimer may include a primer dimer. The primer dimer is unintended products of nucleic acid amplification reaction such as PCR, caused by primer amplification. The primer dimer may inhibit the amplification of a target nucleic acid sequence in an amplification reaction and interfere with accurate analysis. Two types of primer dimer can be formed in PCR reactions. (i) a primer dimer formed when identical primers bind to each other (e.g., Homodimer). In this case, the complementarity between the identical primers is involved in dimer formation and synthesis. (ii) a primer dimer formed when two different primers bind with each other (e.g., Heterodimer). In this case, the forward and reverse primers share some of the regions or nucleotides, bind and amplify in the PCR reaction.

In an embodiment, hybridization between two primers may generate various types of dimers. These dimers may be divided into three categories depending on whether each primer is extended or not. A first category may include a dimeric form in which both the first primer and the second primer can be extended. A second category may include a dimeric form in which only one of the first primer and the second primer can be extended. A third category may include a dimeric form in which neither the first primer nor the second primer can be extended. Also, the category may include dimeric forms in which the first primer and the second primer are partially hybridized (partially overlapped) through 3′-dimer-forming portions of the two primers. Such dimeric form is referred to as a partial dimer. Each primer of the partial dimer may be extended by the polymerization activity of a nucleic acid polymerase. Meanwhile, the dimer of the present disclosure is not limited thereto, and may be broadly interpreted to encompass various types of dimers known to those of skill in the art.

FIG. 1 is a block diagram illustrating a computer device according to an embodiment.

The computer device 100 according to an embodiment of the present disclosure may include a memory 110, a communication unit 120 and a processor 130.

The configuration of a computer device 100 illustrated in FIG. 1 is merely a simplified example. In an embodiment, the computer device 100 may include other configurations for performing a computing environment of the computer device 100, and only some of the disclosed configurations may also configure the computer device 100.

The computer device 100 may mean a node configuring a system for implementing exemplary embodiments of the present disclosure. The computer device 100 may mean a predetermined type of user terminal or a predetermined type of server. The foregoing components of the computer device 100 are illustrative, and some may be excluded, or additional components may be included. For example, when the computer device 100 includes a user terminal, an output unit (not illustrated) and an input unit (not illustrated) may be included within the range.

The computer device 100 may perform technical features according to embodiments of the present disclosure described below. For example, the computer device 100 may provide a prediction result as to occurrence and/or non-occurrence of a dimer of oligonucleotides used in a nucleic acid amplification reaction.

The memory 110 may store at least one instruction executable by the processor 130. In an embodiment, the memory 110 may store a predetermined type of information generated or determined by the processor 130 and a predetermined type of information received by the computer device 100. According to the exemplary embodiment of the present disclosure, the memory 110 may be a storage medium storing computer software that allows the processor 130 to perform operations according to the exemplary embodiment of the present disclosure. Therefore, the memory 110 may also mean computer readable media for storing software codes required for performing the exemplary embodiments of the present disclosure, data that is the execution target of the code, and a result of the code execution.

In an embodiment, the memory 110 may refer to any type of storage medium. For example, the memory 110 may include at least one type of flash memory, hard disk, multimedia card micro, card type memory (e.g., SD or XD memory etc.), Random Access Memory (RAM), Static Random Access Memory (SRAM), Read-Only Memory (ROM), Electrically Erasable Programmable Read-Only Memory (EEPROM), Programmable Read-Only Memory (PROM), magnetic memory, magnetic disk, and optical disk. The computer device 100 may operate in relation to a web storage performing a storage function of the memory 110 on the Internet. The descriptions of the foregoing memory are merely examples, and the memory 110 used in the present disclosure is not limited to the above examples.

The communication unit 120 may be configured regardless of its communication aspect, and may be configured of various communication networks, such as a Personal Area Network (PAN) and a Wide Area Network (WAN). Further, the communication unit 120 may be operated based on the publicly known World Wide Web (WWW), and may also use a wireless transmission technology used in PAN, such as Infrared Data Association (IrDA) or Bluetooth. For example, the communication unit 120 may take in charge of transmitting and receiving data required to perform a technique according to an embodiment of the present disclosure.

The processor 130 may perform technical features according to embodiments of the present disclosure described below, by executing at least one instruction stored in the memory 110. In an embodiment, the processor 130 may consist of one or more cores, and may include a processor for analyzing and/or processing data, such as a Central Processing Unit (CPU), a General Purpose Graphics Processing Unit (GPGPU), and a Tensor Processing Unit (TPU) of the computer device 100.

The processor 130 may read a computer program stored in the memory 110 to obtain a prediction result for a dimerization of oligonucleotide from a dimer prediction model according to an embodiment of the present disclosure. Here, the dimer prediction model is an Artificial Intelligence (AI) based model learned to predict a dimerization in a nucleic acid amplification reaction. In an embodiment, the computer device 100 may obtain the dimer prediction model through AI-based learning. In an embodiment, the computer device 100 may obtain the dimer prediction model pre-trained by other device, through the communication unit 120 from the other device.

According to an exemplary embodiment of the present disclosure, the processor 130 may perform an operation for learning of a neural network. The processor 130 may perform calculation for learning of the neural network, for example, processing of input data for learning in deep learning (DN), extraction of a feature from the input data, calculation of an error, and updating of a weight of the neural network using backpropagation. At least one of a CPU, a GPGPU, and a TPU of the processor 130 may process the learning of the network function. For example, both the CPU and the GPGPU may process the learning of the network function and data classification using the network function. Further, according to an exemplary embodiment of the present disclosure, processors of a plurality of computing devices are used together to process the learning of the network function and data classification using the network function. Further, the computer program executed in the computing device according to the exemplary embodiment of the present disclosure may be a CPU, GPGPU, or TPU executable program.

The computer device 100 may include any type of user terminal and/or any type of server. The user terminal may include any type of terminal capable of interacting with a server or other computing device.

The user terminal may include, for example, a cell phone, a smart phone, a laptop computer, a personal digital assistants (PDA), a slate PC, a tablet PC, and an ultrabook.

The server may include, for example, any type of computing system or computing device, such as a microprocessor, a mainframe computer, a digital processor, a portable device, a device controller, and the like.

In a further embodiment, the server may refer to an entity that stores and manages a data of a plurality of nucleic acid sequences and/or a sequence data of an oligonucleotide. The server may include storage unit (not illustrated) for storing the data of the plurality of nucleic acid sequences and/or the sequence data of the oligonucleotide. The storage unit may be present inside the server and present under the management of the server. As other example, the storage unit may present outside the server and may be implemented in a form capable of communicating with the server. In this case, the storage unit may be managed and controlled by another external server different from the server.

According to an exemplary embodiment of the present disclosure, the computer device 100 may obtain a dimer prediction model learned to predict a dimerization in a nucleic acid amplification reaction.

First, a basic structure and an operation of the dimer prediction model as an AI-based model are briefly described below. Specific structures and operations such as learning method will be described later with reference to the drawings.

The dimer prediction model in the present disclosure may refer to any type of computer programs that operates based on a network function, artificial neural network and/or neural network. Throughout the specification, a model, a network function, and a neural network may be used to have the same meaning. The neural network may generally be configured by a set of interconnected calculating units which may be referred to as “nodes”. The “nodes” may also be referred to as “neurons”. The neural network is configured to include at least one node. The nodes (or neurons) which configure the neural networks may be connected to each other by one or more “links”.

In the neural network, one or more nodes connected through the link may relatively form a relation of an input node and an output node. Concepts of the input node and the output node are relative so that an arbitrary node which serves as an output node for one node may also serve as an input node for the other node and vice versa. As described above, an input node to output node relationship may be created with respect to the link. One or more output nodes may be connected to one input node through the link and vice versa.

In the input node and output node relationship connected through one link, a value of the output node may be determined based on data input to the input node. The node which connects the input node and the output node to each other may have a weight. The weight may be variable and may vary by the user or the algorithm to allow the neural network to perform a desired function. For example, when one or more input nodes are connected to one output node by each link, the output node may determine an output node value based on values input to the input nodes connected to the output node and a weight set to the link corresponding to the input nodes.

As described above, in the neural network, one or more nodes are connected to each other through one or more links to form an input node and output node relationship in the neural network. In the neural network, a characteristic of the neural network may be determined in accordance with the number of the nodes and links and a correlation between the nodes and links, and a weight assigned to the links. For example, when there are two neural networks in which the same number of nodes and links are provided and weights between links are different, it may be recognized that the two neural networks are different.

The neural network may be configured to include a set of one or more nodes. A subset of nodes configuring the neural network may configure a layer. Some of the nodes which configure the neural network may configure one layer based on distances from the initially input nodes. For example, a set of nodes whose distance from the initially input node is n may configure n layers. The distance from the initially input node may be defined by a minimum number of links which need to go through to reach from the initially input node to the corresponding node. However, the definition of the layer is arbitrary provided for description and the dimensionality of the layer in the neural network may be defined differently from the above description. For example, the layer of the nodes may be defined by a distance from the finally output node.

The initially input node may refer to one or more nodes to which data is directly input without passing through the link in the relationship with other nodes, among the nodes in the neural network. Alternatively, in the neural network, in the relationship between nodes with respect to the link, the initially input node may refer to nodes which do not have other input nodes linked by the link. Similarly, the final output node may refer to one or more nodes which do not have an output node, in the relationship with other nodes, among the nodes in the neural network. Further, a hidden node may refer to nodes which configure the neural network, other than the initially input node and the finally output node.

In the neural network according to an exemplary embodiment of the present disclosure, the number of nodes of the input layer may be equal to the number of nodes of the output layer and the number of nodes is reduced and then increased from the input layer to the hidden layer. Further, in the neural network according to another exemplary embodiment of the present disclosure, the number of nodes of the input layer may be smaller than the number of nodes of the output layer and the number of nodes is reduced from the input layer to the hidden layer. Further, in the neural network according to another exemplary embodiment of the present disclosure, the number of nodes of the input layer may be larger than the number of nodes of the output layer and the number of nodes is increased from the input layer to the hidden layer. The neural network according to another exemplary embodiment of the present disclosure may be a neural network obtained by the combination of the above-described neural networks.

A deep neural network (DNN) may refer to a neural network including a plurality of hidden layers in addition to the input layer and the output layer. When the deep neural network is used, latent structures of the data may be identified. The deep neural network may include a convolutional neural network (CNN), a recurrent neural network (RNN), auto encoder, a generative adversarial network (GAN), a restricted Boltzmann machine (RBM), a deep belief network (DBN), a Q network, a U network, and a Siamese network. Description of the above-described deep neural networks is only an example, and the present disclosure is not limited thereto.

The neural network may be learned by at least one of supervised learning, unsupervised learning, semi supervised learning, self-supervised learning or reinforcement learning methods. The learning of the neural network may refer to a process of applying knowledge for the neural network to perform a specific operation to the neural network.

The neural network may be learned to minimize an error of the output. Training data is repeatedly input to the neural network during the learning of the neural network, an output of the neural network for the training data and an error of the target are calculated, and an error of the neural network is back-propagated from the output layer of the neural network to the input layer direction so as to reduce the error to update a weight of each node of the neural network. In the case of the supervised learning, training data labeled with a correct answer (that is, labeled training data) may be used for each training data, but in the case of the unsupervised learning, training data not labeled with a correct answer (that is, unlabeled training data) may be used for each training data. That is, for example, the training data of the supervised learning for data classification may be training data labeled with category. The labeled training data is input to the neural network and the error may be calculated by comparing the output (category) of the neural network and the label of the training data. As another example, in the case of the unsupervised learning for data classification, an error may be calculated by comparing the training data which is an input with the neural network output. The calculated error is backpropagated to a reverse direction (that is, a direction from the output layer to the input layer) in the neural network and a connection weight of each node of each layer of the neural network may be updated in accordance with the backpropagation. A variation of the connection weight of the nodes to be updated may vary depending on a learning rate. The calculation of the neural network for the input data and the backpropagation of the error may configure a learning epoch. The learning rate may be differently applied depending on the repetitive number of the learning epochs of the neural network. For example, at the beginning of the neural network learning, the neural network quickly ensures a predetermined level of performance using a high learning rate to increase efficiency and at the late stage of the learning, the low learning rate is used to increase the precision.

In the learning of the neural network, normally, the training data may be a subset of the actual data (that is, data to be processed using the learned neural network). Therefore, there may be a learning epoch that the error of the training data is reduced, and the error is increased for the actual data. The overfitting is a phenomenon in which the training data is excessively learned so that an error for real data is increased. The overfitting may act as a cause of the increase of the error of the machine learning algorithm. Various optimization methods may be used to prevent the overfitting. In order to prevent the overfitting, a method of increasing training data, regularization, a dropout method which omits some nodes of the network during the learning process, or use of batch normalization layers may be applied.

In an embodiment, the dimer prediction model may include at least a portion of a transformer. The transformer may comprise an encoder encoding embedded data and a decoder decoding encoded data. The transformer may have a structure that receives a series of data and outputs a series of data of different type through encoding and decoding steps. In an embodiment, the series of data may be processed into a form computable by the transformer. A process of processing the series of data into the form computable by the transformer may include an embedding process. Expressions such as a data token, an embedding vector, and an embedding token may refer to embedded data in a form that can be processed by the transformer.

In order for the transformer to encode and decode a series of data, encoders and decoders in the transformer may be processed using an attention algorithm. The attention algorithm refers to an algorithm that obtains a similarity of one or more keys for a given query, applies the obtained similarity to a value corresponding each key, and then calculates an attention value by weighted sum.

Various types of attention algorithms may be classified, according to a method of setting a query, a key, and a value. For example, when one attention algorithm obtains an attention by setting the query, the key and the value all the same, the one attention algorithm may refer to a self-attention algorithm. When one attention algorithm obtains an attention by reducing a dimension of an embedding vector and obtaining individual attention heads in order to process a series of input data in parallel, the one attention algorithm may refer to a multi-head attention algorithm.

In an embodiment, the transformer may comprise modules performing a plurality of multi-head self-attention algorithms or multi-head encoder-decoder algorithms. In an embodiment, the transformer may include additional components other than the attention algorithm, such as embedding, normalization, and SoftMax. A method for constructing the transformer using the attention algorithm may include a method disclosed in Vaswani et al., Attention Is All You Need, 2017 NIPS, which is incorporated herein by reference.

The transformer may be applied to various data domains such as embedded natural language, segmented image data, and audio waveforms to convert a series of input data into a series of output data. In order to convert data having various data domains into a series of data that can be input to the transformer, the transformer may perform embedding the data. The transformer may process additional data representing a relative positional relationship or phase relationship between a set of input data. Alternatively, vectors representing a relative positional relationship or phase relationship between input data may be additionally applied to a series of input data, and the series of input data may be embedded. In an example, the relative positional relationship between the series of input data may include word order in a natural language sentence, relative positional relationship of each segmented image, and temporal sequence of segmented audio waveforms, but is not limited to. A process for adding a data representing the relative positional relationship or phase relationship between the series of input data may refer to positional encoding.

In an embodiment, the transformer may include a Recurrent Neural Network (RNN), a Long Short-Term Memory (LSTM) network, a Bidirectional Encoder Representations from Transformers (BERT), or a Generative Pre-trained Transformer (GPT).

According to an embodiment of the present disclosure, the dimer prediction model may be learned by a transfer learning method. In an embodiment, a process of the transfer learning for the dimer predictive model may be implemented by one entity such as by one entity, such as a method in which the process is performed by a server. In an embodiment, the process of the transfer learning for the dimer predictive model may be implemented by a plurality of entities, such as a method in which a part of the process is performed by a user terminal and another part of the process is performed by a server.

Here, the transfer learning refers to a learning method in which a pre-trained model having a first task is obtained by pre-training using a large amount of unlabeled training data in a semi-supervised learning method or self-learning method and a targeted model is implemented by fine-tuning the pre-trained model to be suitable for a second task using a labeled training data in a supervised learning method.

In an embodiment, the first task of the pre-training and the second task of the fine-tuning may be different. As a specific example, the first task may be for language modeling of a phenomenon of a sequence pattern similar to a kind of language represented in a nucleic acid sequence of a target analyte (e.g., a virus). Alternatively, the first task may be a general-purpose task using the nucleic acid sequence of the target analyte. Also, the second task may be a sub-task of the first task and may be to predict a dimerization of an oligonucleotide in a nucleic acid amplification reaction. For example, a pre-trained language model may be obtained by training using training data including nucleic acid sequences of various viruses based on a language model to be suitable for a task for language modeling the sequence pattern of the virus. And then, a dimer prediction model may be implemented by fine-tuning a structure and weights of the pre-trained language model to be suitable for dimer prediction.

In an embodiment, the dimer prediction model learned by the transfer learning method may refer to a model in which an output of a dimerization probability value in a plurality of oligonucleotides is applied, as the fine-tuning method, to a model pre-trained using type data and order data of bases. In an embodiment, the pre-trained model may refer to a deep-learning language model learned according to a specific task (e.g., classification, detection, segmentation, etc.) or a general-purpose task. For example, the pre-trained model may be pre-trained based on types of bases (e.g., A, G, C, and T) and the arrangement order of bases.

In an embodiment, the fine-tuning may refer to a method for modifying an architecture to be suitable for a new task (e.g., predicting a dimerization) based on a pre-trained model and updating learning from weights of the pre-trained model. As an example, the fine-tuning may include a process in which parameters of the pre-trained model are updated by additionally training the pre-trained model using specific data.

In an embodiment, the fine-tuning may include additionally training the pre-trained model to be suitable for predicting a dimerization. For example, the fine-tuning may include a concept of post training a pre-trained model by transferring a task of the pre-trained model to a specific or different task.

As described above, since the transfer learning uses the pre-training and the fine-tuning, it has advantage of implementing high performance even when using a relatively small amount of labeled training data.

Hereinafter, various embodiments of a process in which the dimer prediction model presented above is learned by the transfer learning method will be described in more detail.

### Examples of Pre-Training

The computer device 100 may obtain a pre-trained model. In an embodiment, the computer device 100 may obtain a pre-trained model by performing a pre-training of a plurality of nucleic acid sequences. In an embodiment, the computer device 100 may receive a model pre-trained by another device from another device (or storage unit) through network.

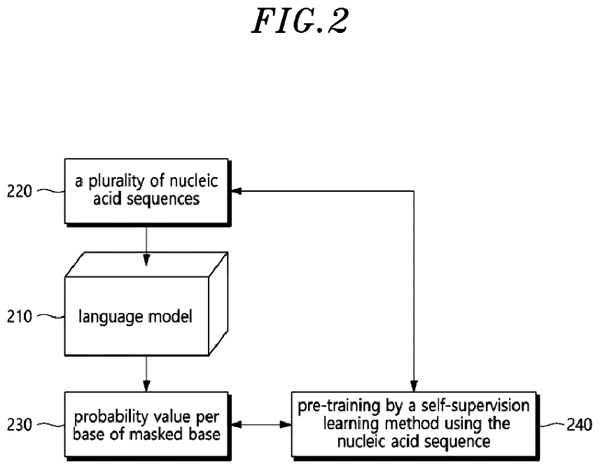

FIG. 2 is a view illustrating a concept of pre-training process according to an embodiment. In an embodiment, FIG. 2 illustratively describes a method in which the pre-training is performed using a language model 210. A language model 210 performed pre-training may correspond to the pre-trained model in the present disclosure. In addition, above-mentioned examples of the basic structure and operation of the dimer prediction model, such as a neural network and learning using the neural network, may be applied to the language model 210.

Referring to FIG. 2, the computer device 100 may perform a process of pre-training using the language model 210. In an embodiment, the language model 210 is an artificial neural network, and may comprise at least a part of the above-described transformer. As a specific example, the language model 210 may include BERT or GPT, which are transformer-based language models.

In an embodiment, a plurality of nucleic acid sequences 220 may be used as a training data in the process of pre-training. Here, the plurality of nucleic acid sequences 220 refer to nucleic acid sequences of an organism, for example, an arrangement of bases or base pairs in a gene of an organism. In an embodiment, the plurality of nucleic acid sequences 220 may be obtained from a specific group of organisms. For example, the specific group may include organisms (e.g., viruses, bacteria, human, and etc.) belonging to any one hierarchical level in which a target analyte is located in a biological classification system (or taxonomy) having a hierarchical structure. For example, the plurality of nucleic acid sequences 220 may be sequences of Sars-CoV2, Corona family, RNA virus or viruses.

In an embodiment, a data of the plurality of nucleic acid sequences 220 may be obtained from a database. For example, the computer device 100 may gather a large amount of virus sequences by accessing public database such as National Center for Biotechnology Information (NCBI) and Global Initiative for Sharing All Influenza Data (GISAID). Further, the computer device 100 may perform a text preprocessing the gathered virus sequences to process into training data for pre-training.

In an embodiment, the plurality of nucleic acid sequences 220 may be classified according to a plurality of groups for a target analyte or an organism. Further, training data comprising the plurality of nucleic acid sequences 220 corresponding to each group may be provided to each of a plurality of language model 210.

In an embodiment, the language model 210 may be learned to predict a probability value 230 per base of masked base, based on a type and an order of bases in the plurality of nucleic acid sequences 220. Specifically, the language model 210 may be trained by a semi-supervised learning method performed in which a mask to some of bases in the nucleic acid sequences is applied and then an answer of the masked base is found in the process of pre-training. For example, the language model 210 may be pre-trained by a self-supervision learning method 240 performed in which an arbitrarily determined base from the nucleic acid sequence 220 as a training input data is masked and a task to predict the masked base is assigned, without a label of answer (that is, training answer data).

For example, the language model 210 may assign a probability to a sequence of bases included in the plurality of nucleic acid sequences 220, consider which bases appear before and after the masked base, and output a probability value 230 per base by assuming an occurrence probability of multiple bases that can be a masked base candidate. In addition, the language model 210 may calculate an error by comparing the probability value 230 per base of the masked base, which is output data, and types and order of bases included in the plurality of nucleic acid sequences 220 of the training data. Further, parameters of the language model 210 may be updated according to backpropagation for reducing errors.

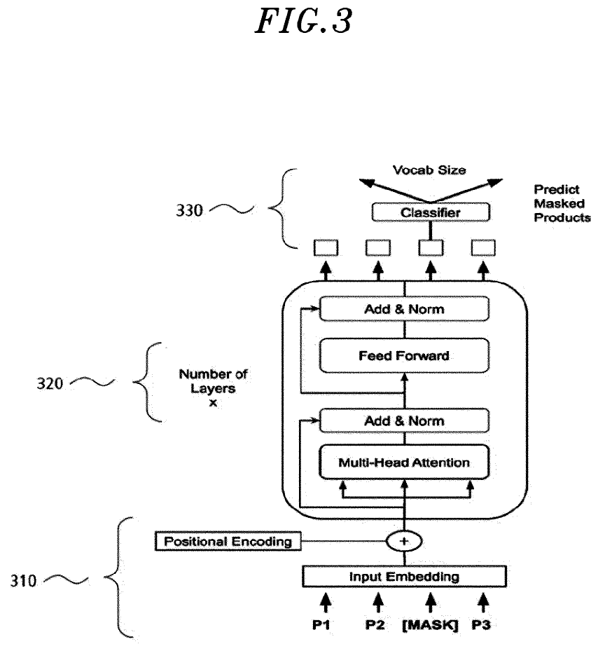

FIG. 3 is a view illustrating a structure and an operation of a BERT-based language model 210 in the pre-training process according to an embodiment. As a specific embodiment, at least a part of BERT using a structure in which a plurality of encoders encoding embedded data are connected may be used for the pre-training using the language model 210.

Referring to FIG. 3, the language model 210 may refer to a classification model that outputs a plurality of prediction values for each of masked tokens using the masked tokens and non-masked tokens corresponding to the plurality of nucleic acid sequences 220. Here, one prediction value may correspond to one class of the language model 210. As an example, the language model 210 may receive the masked tokens and non-masked tokens as the nucleic acid sequences 220. As another example, the language model 210 may receive the nucleic acid sequences 220 as input and perform preprocessing the input nucleic acid sequences 220 to generate the masked tokens and non-masked tokens.

As shown in the FIG. 3, the language model 210 may include at least one of an input embedding layer 310, an encoder layer 320 and a first classifier layer 330.

In an embodiment, the input embedding layer 310 may convert the plurality of nucleic acid sequences 220, which are a series of input data, into a form computable by an encoder. In an embodiment, the input embedding layer 310 may include at least one of a token embedding layer for tokenizing bases in the nucleic acid sequences 220, and a position (or positional) embedding layer for applying a position data to vectors. In addition, according to embodiments, the input embedding layer 310 may further include an additional embedding layer such as a segment embedding layer.

In an embodiment, the token embedding layer may perform a tokenization process that the nucleic acid sequences 220 are tokenized with tokens each having two or more bases. In an embodiment, the tokenization process may refer to an operation of grouping a plurality of bases included in the nucleic acid sequences. Each one of the tokens generated in the tokenization process may include one or more bases.

In an embodiment, the tokens may comprise each bases tokenized by (i) dividing the nucleic acid sequences by k unit (wherein k is a natural number) or (ii) dividing the nucleic acid sequences by a function unit. For example, a k-mer technique in which k bases are divided by k unit may be used in the tokenization process. In an example using the k-mer technique, if k is 3, a number of bases in each token may be three in total. For another example, a gene prediction technique in which the nucleic acid sequences are divided according to a function of splicing may be used in the tokenization process. As a specific example, the dividing by the function unit may include at least one of dividing by codon unit capable of coding one amino acid and dividing by section unit related to such as gene expression (e.g., transcription, translation) or expression pattern.

In a further embodiment, the tokenization process may be performed based on various techniques. As an example, a tokenization of a nucleic acid sequence may be performed based on a Bite Pair Encoding algorithm. As an example, a nucleic acid sequence may be tokenized based on an optimal k-mer size. In this example, since each organism may have a specific k-mer size that may best represent its genome, a tokenization may be performed based on the specific k-mer size determined in organism unit. As an example, a tokenization may be performed based on a DNA motif. As an example, a tokenization of a nucleic acid sequence may be performed based on an exon, which is a unit of a coding region transcribing RNA within a gene of higher organisms.

In an embodiment, the token embedding layer may generate masked tokens and non-masked tokens by performing preprocessing a plurality of tokens generated through the tokenization process. For example, the token embedding layer may generate the masked tokens by masking at least one of a plurality of non-masked tokens tokenized from a nucleic acid sequence with a special toke, called [MASK] token.

In an embodiment, the token embedding layer may represent each token as a vector, for example, may convert the tokenized bases into an embedding vector in form of a dense vector by word embedding the tokenized bases.

In an embodiment, the position (or positional) embedding layer may apply a position data to the embedding vectors before the embedding vectors are used as an input of an encoder. For example, a position embedding for obtaining the position data through learning may be used. For example, a method for learning a plurality of position embedding vectors corresponding to a length of a nucleic acid sequence and adding the corresponding position embedding vector to each embedding vector may be used.

Embedded data processed into a form computable by an encoder while passing through the above-described input embedding layer 310 may be provided as an input to the encoder layer 320 having a structure in which a plurality of encoders are stacked. Accordingly, the result of calculating data embedded in the first encoder in the encoder layer 320 is output toward the next encoder, and a context vector generated comprehensively considering the input embedded data may be output in the last encoder.

In an embodiment, the encoder layer 320 may include N (e.g., 12, 24, etc.) encoder blocks. A structure in which N encoder blocks are stacked means that a meaning of an entire input sequence is repeatedly constructed N times. The larger a number of encoder blocks, the better a semantic relationship between bases in a nucleic acid sequence may be reflected. N encoder blocks may be configured in a form in which the entire input sequence is recursively processed.

In an embodiment, each encoder block may output a weight-based calculation result for provided input using a multi-head attention algorithm. For example, each encoder block may output a concatenation which is a result of calculating an attention h time using different weight matrices and connecting them together. As a result, even small differences may lead to significant differences in results. Further, learning effect may be improved as inputs and processing results in each encoder block are calculated through normalization, residual connection, and feed-forward neural network.

In an embodiment, the first classifier layer 330 may process a result output from the encoder layer 320 into a form of data meaningful to the user. For example, the first classifier layer 330 may include a classifier that performs a classification function for performing the first task by using an embedding vector (e.g., context vector) output from the last encoder block of the encoder layer 320 as an input. For example, the classifier may include a SoftMax function for outputting the probability value 230 per base of the masked token by using an output embedding vector corresponding to a position of the masked token as an input.

An error calculation and a weight update may be performed by comparing an output of the first classifier layer 330 with the bases included in the nucleic acid sequence of the training data. As such the pre-training proceeds, the pre-trained model may be obtained. Since the pre-learning using a Masked Language Modeling (MLM) method for finding an answer of the masked base predicts the masked bases in consideration of bases located in bidirection within a given nucleic acid sequence, prediction accuracy is high. Further, a pre-trained model with a better understanding of a pattern of nucleic acid sequences having characteristics similar to a kind of language may be implemented.

In an embodiment, a method for learning to find an answer of the masked base and a method for learning to correct an incorrect base after replacing some bases with other bases may be used together in the process of the pre-training. In addition, Next Sentence Prediction (NSP) may be further used in the process of the pre-training. For example, the pre-training using both the MLM method and the NSP method may be performed.

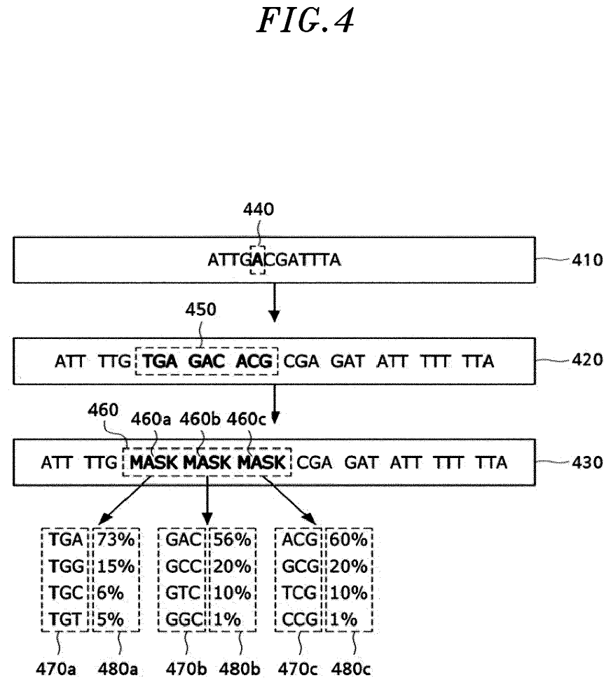

FIG. 4 is a view illustrating an example method for predicting probabilities per base by the pre-trained model in the computer device 100 according to an embodiment.

FIG. 4 illustrates that a number of bases in one token or one masked token is three. Depending on the implementation, it would be apparent to those skilled in the art that the number of bases in a token may include a various number.

In an embodiment of the present disclosure, a nucleic acid sequence 410 may be obtained. The nucleic acid sequence 410 may include, as a sequence representing a specific species, a nucleic acid sequence first discovered for the specific species, or a nucleic acid sequence occupied the most proportion in the specific species. In an additional embodiment, the nucleic acid sequence 410 may include a nucleic acid sequence corresponding to a variant of the specific species rather than a sequence representative of the specific species, depending on the implementation.

In an embodiment of the present disclosure, the pre-trained model may calculate the probability value 230 per base of a specific base 440 in the nucleic acid sequence 410. When the nucleic acid sequence 410 is provided to the pre-trained model, the pre-trained model may determine at least one specific base 440 from the nucleic acid sequence 410 and output the probability value 230 per base of the specific base 440.

In an example shown in FIG. 4, the base 440 for calculating the probability value 230 per base in the nucleic acid sequence 410 is A. The pre-trained model may tokenize the nucleic acid sequence 410 consisting of a plurality of bases into a plurality of tokens 420.

FIG. 4 illustrates that each of the plurality of tokens 420 is generated according to a 3-mer technique. Each of the plurality of tokens 420 may include three bases. In an example in FIG. 4, a first base, A, in the nucleic acid sequence 410 may correspond to a token comprising ATT. A second base, T, in the nucleic acid sequence 410 may correspond to a token comprising ATT and a token comprising TTG. A third base, T, in the nucleic acid sequence 410 may correspond to a token comprising ATT, a token comprising TTG, and a token comprising TGA. A fourth base, G, may correspond to a token comprising TTG, a token comprising TGA, and a token comprising GAC. As such, FIG. 4 shows that tokens are generated while moving one base in the arrangement order of a plurality of bases in the nucleic acid sequence 410. In this example, two bases may be shared with each other for adjacent tokens.

In an embodiment, each of the generated tokens may correspond to a k-mer resulting from dividing bases in the nucleic acid sequence 410 by k unit. Here, k may be a natural number, for example, k may refer to a natural number not less than 3 and not more than 20. In this example, a number of bases in each of the tokens may correspond to k. That is, when k is 3, one token may include 3 bases.

In an embodiment, each of the masked tokens may include k bases. In addition, a count of the masked tokens generated corresponding to each of the bases in a range of kth to n-kth among the n bases comprised in the nucleic acid sequence may correspond to k. Here, each of k and n is a natural number, for example, k≥2 and n≥2k.

For example, among the plurality of tokens 420, the tokens 450 corresponding to the first base 440, A, of the nucleic acid sequence 410 may include TGA, GAC, and ACG. The tokens 450 may include a first token TGA including the first base 440 at a first position, a second token GAC including the first base 440 at a second position, and a third token ACG including the first base 440 A at a third position. In the example described above, since tokens are generated based on the 3-mer technique, one token may include three bases and a total of three tokens may correspond to one base.

In an embodiment, for the first base 440 of the plurality of bases 410, a first set of tokens 450 including the first base 440 at different positions and a first set of masked tokens 460 (460a, 460b, and 460c) corresponding to the first set of tokens 450 may be generated. In an embodiment, the probability value 230 per base of the first base 440 may be determined based on prediction values (480a, 480b, and 480c) output from the language model 210 for each of the first set of masked tokens 460 (460a, 460b, and 460c).

The pre-trained model may obtain the masked tokens 460 by applying a mask to the first set of tokens 450 that are at least some tokens of the plurality of tokens 420. For example, the pre-trained model may generate the masked tokens 460 by applying a mask to each of three tokens corresponding to the first base 440.

In the example described above, since tokens are generated based on the 3-mer technique, one masked token may correspond to three bases, and a count of masked tokens may also correspond to three.

The pre-trained model may generate an intermediate input data 430 from the tokens 420. In an embodiment, the intermediate input data 430 may include the masked tokens 460 and non-masked tokens.

In an example, the pre-trained model may obtain prediction values (480a, 480b, and 480c) of classes (470a, 470b, and 470c) for each of the masked tokens 460 (460a, 460b, and 460c). The pre-trained model may calculate the probability value 230 per base of the first base 440 based on the obtained prediction values (480a, 480b, and 480c). For example, in an embodiment, the pre-trained model may calculate the probability value 230 per base using an average of the predicted values.

In an embodiment, parameters of the pretrained model may be updated to minimize errors, by comparing the probability value 230 of the specific base 440 with the specific base 440 in the nucleic acid sequence 410, or by comparing prediction values (480a, 480b, 480c) of the masked tokens 460 (460a, 460b, and 460c) with tokens 450 corresponding to the specific base 440. In this way, the pre-trained model may be trained repeatedly to find an answer of the masked base better.

### Examples of Fine-Tuning

The computer device 100 may obtain a dimer prediction model by fine-tuning a pre-trained model. In an embodiment, the fine-tuning may comprise determining a structure of a dimer prediction model using the pre-trained model and training the dimer prediction model using a training data for dimer prediction.

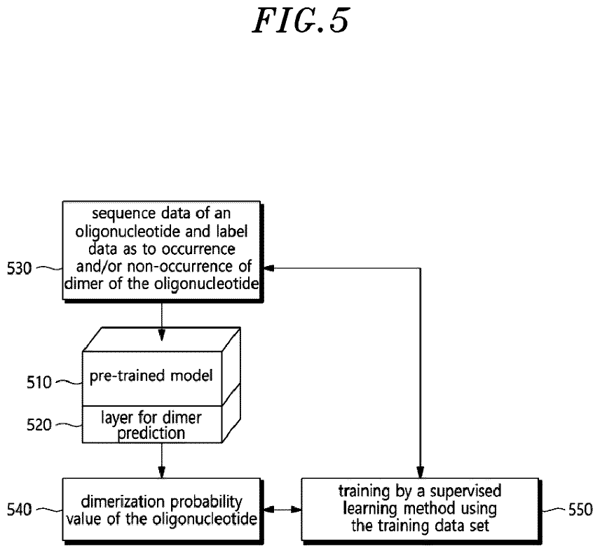

FIG. 5 is a view illustrating a concept of fine-tuning process according to an embodiment. In an embodiment, FIG. 5 illustratively describes a method in which the fine-tuning is performed using the pre-trained model 510. As described above, the pre-trained model 510 may correspond to the language model 210 having undergone pre-training.

Referring to FIG. 5, a process of fine-tuning may be performed using the pre-trained model 510. As described above, the pre-trained model 510 according to an embodiment may refer to a model pre-trained with a task different from a task for predicting a dimerization of an oligonucleotide. The pre-trained model 510 according to another embodiment may refer to a model pre-trained with a general-purpose task.

In an embodiment, a structure of the dimer prediction model may be determined using the pre-trained model 510 in the process of fine-tuning. For example, the structure of the dimer prediction model may be determined by importing the embedding layer 310 and the encoder layer 320 from the pre-trained model 510 whose weights have been already calculated through the pre-training and then adding a layer for dimer prediction 520 to a last encoder block of the encoder layer 320. Depending on the implementation, the pre-trained model 510 having undergone the fine-tuning and the layer for dimer prediction 520 may correspond to the dimer prediction model.

In an embodiment, the fine-tuning may be performed using a plurality of training data sets. A sequence data of an oligonucleotide and a label data as to occurrence and/or non-occurrence of dimer of the oligonucleotide 530 may be used as the training data set.

In an embodiment, each training data set may comprise (i) a training input data comprising a sequence data of two or more oligonucleotides and (ii) a training answer data comprising a label data as to occurrence and/or non-occurrence of dimer of the two or more oligonucleotides. In this case, the occurrence and/or non-occurrence of dimer may indicate whether a dimer is formed between the two or more oligonucleotides, for example, may be expressed as a label as to occurrence and or non-occurrence of a pair-dimer.

In another embodiment, each training data set may comprise (i) a training input data comprising a sequence data of one or more oligonucleotides and (ii) a training answer data comprising a label data as to occurrence and/or non-occurrence of dimer of the one or more oligonucleotides. In this case, the occurrence and/or non-occurrence of dimer may indicate whether a dimer is formed within the one or more oligonucleotides, for example, may be expressed as a label as to occurrence and or non-occurrence of a self-dimer or a pair-dimer.

Here, oligonucleotides used in the process of the fine-tuning refer to oligonucleotides used in supervised learning for dimer prediction. In addition, a sequence of an oligonucleotide means a sequence of bases arranged in order as a component of an oligonucleotide.

In an embodiment, the sequence data of the oligonucleotide may comprise a first sequence (e.g., a forward sequence) and a second sequence (e.g., a reverse sequence). In an embodiment, the first sequence may be a forward sequence (or a reverse sequence) of the first oligonucleotide and the second sequence may be a forward sequence (or a reverse sequence) of a second oligonucleotide. The first oligonucleotide and the second oligonucleotide may be the same or different oligonucleotides. For example, the sequence data of the oligonucleotide may comprise various combinations of sequences, such as, a forward primer sequence and a reverse primer sequence of one pair primer, a forward primer sequence and a forward primer sequence of different pair primers, or a reverse primer sequence and a reverse primer sequence of different pair primers.

Here, the forward sequence may include a sequence of a forward primer, for example, may include a sequence of a primer acting as a point of initiation of a coding or positive strand of a target analyte. In addition, the reverse sequence may include a sequence of a reverse primer, for example, may include a sequence of a primer acting as a point of initiation for synthesizing a complementary strand of the coding sequence or non-coding sequence of the target analyte.

In an embodiment, the sequence data of the oligonucleotide may comprise at least one of a third sequence to a Lth sequence (L is a natural number not less than 4). Similarly, the third sequence may be a forward sequence (or a reverse sequence) of a third oligonucleotide and the Lth sequence may be a forward sequence (or a reverse sequence) of a Lth oligonucleotide. Each oligonucleotide may be the same or different from other oligonucleotides.

Table 1 illustratively shows the plurality of training data sets used in the fine-tuning according to an embodiment. As shown in Table 1, each training data set may comprise inputs as to the first sequence and the second sequence, and a label as to whether a dimerization between the first sequence and the second sequence occurs or not (e.g., a label ‘1’ corresponds to ‘occurrence of a dimerization’ and a label ‘0’ corresponds to ‘non-occurrence of a dimerization’). In an embodiment, the sequences and the label of each training data set may be obtained from experimental data of a nucleic acid amplification reaction for each sample using oligonucleotides having the sequences.

TABLE 1

| | First sequence | Second sequence | Label |
|---|---|---|---|
| Set1 | AGCATTGTGGGTAGTAAGGTATAAA | AGCTCAAAATCTACATAACCCCTC | 1 |
| Set2 | AGCGTTATTGTTGAGAAATGGATTG | AGCACAAAAAAATTTATACAAAAAACAACT | 0 |
| ... | ... | ... | ... |
| SetN-1 | AGCGTGGTTATTGGATGGGTTTG | AGCAAATCTTTACTAAAAAAAATTTACCTT | 1 |
| SetN | AGCTGTTTTTTTTTTTGTTGTGGGTAA | AGCCTATAAATCCTAATACTTAACTCA | 0 |

The first sequence and the second sequence of each training data set in shown in Table 1 may refer to a primer pair designed for determining a pre-determined amplification region of target nucleic acid sequence or non-paired primers. In an embodiment, each training data set may comprise a sequence set comprising at least one primer pair (e.g., a forward sequence and a reverse sequence) and/or at least one non-paired primer (or probe).

In an embodiment, the plurality of training data sets used in the fine-tuning may be a result of a dimer experiment on each sample or its processed data, transformed data or separated data. For example, the dimer experiment may be performed in a method in which a nucleic acid amplification reaction is performed in each reaction well containing oligonucleotides having different base sequences and then a signal indicating occurrence and/or non-occurrence of dimer of oligonucleotides in each reaction well may be detected. In an embodiment, as the signal detection method indicating occurrence of amplification of a target analyte, a fluorescence signal detection and analysis may be used. In an embodiment, the result of the dimer experiment may be separately stored and managed in the storage unit and loaded from the storage unit in the process of the fine-tuning. As an example, the computer device 100 may receive a plurality of dimer experiment results from the storage unit, another device or storage medium. Further, the computer device 100 may extract (i) sequences of oligonucleotides and (ii) occurrence and/or non-occurrence of dimer in the experiment from each dimer experiment result. Further, using the extracted result, the computer device 100 may generate each training data set, which comprises (i) a training input data comprising a sequence data of the corresponding oligonucleotides and (ii) a training answer data comprising a label data as to the occurrence and/or non-occurrence of dimer. Alternatively, the computer device 100 may receive the plurality of training data sets from the storage unit, another device or storage medium.

In an embodiment, the number of the training data sets used in the fine-tuning may be less than the number of the training data used in the pre-training. For example, where a large amount of training data such as hundreds of thousands to millions of training data is used for the pre-training, a smaller amount of training data such as a thousand to several thousand of training data may be used for the fine-tuning.

In an embodiment, the dimer prediction model may be learned by a method in which the pre-trained model 510 is fine-tuned to output a dimerization probability value 540 of the oligonucleotides using the above-described training data set. More specifically, the dimer prediction model may output the dimerization probability value 540 of the oligonucleotides, based on a type and order of bases included in each sequence data of oligonucleotides which is the training input data in the process of the fine-tuning. In addition, the dimer prediction model may be trained by a supervised learning method 550 using the training data set as labeled data in the fine-tuning.

For example, an error may be calculated by comparing the dimerization probability value 540 which is an output data of the dimer prediction model with a label data as to occurrence and/or non-occurrence of dimer labeled as an answer to the sequence data of oligonucleotides. Further, parameters of the dimer prediction model may be updated according to a backpropagation method for reducing the error.

According to an embodiment, the fine-tuning may comprise (i) joining sequences of the two or more oligonucleotides by using a discrimination token and (ii) tokenizing the joined sequences to obtain a plurality of tokens. As a specific example, when the dimer prediction model includes a BERT, tokenized inputs in a form that the encoders in the BERT can process should be provided to a plurality of the encoders in the BERT. For this purpose, one joined sequences may be generated by joining sequences of the oligonucleotides for which dimerization is to be predicted, and then tokenized to provide a plurality of tokens that may be input to a plurality of the encoders.

According to an embodiment, the fine-tuning may further comprise (iii) predicting a dimerization of the two or more oligonucleotides using a context vector generated from the plurality of tokens, and (iv) training the pre-trained model for reducing a difference between the predicted result and the training answer data. In the example where the dimer prediction model includes a BERT, as the plurality of tokens are input to the plurality of encoders in the BERT, a context vector in which the sequences of the two or more oligonucleotides are comprehensively considered may be output as a compressed vector representation. In addition, this context vector may be input to a classifier connected to the encoder, and a classification value or a probability value as the predicted result may be output from the classifier. Further, an error may be calculated by comparing the predicted result with the label data as to occurrence and/or non-occurrence of dimer in the training answer data, and parameters of the dimer prediction model may be updated for reducing the error.

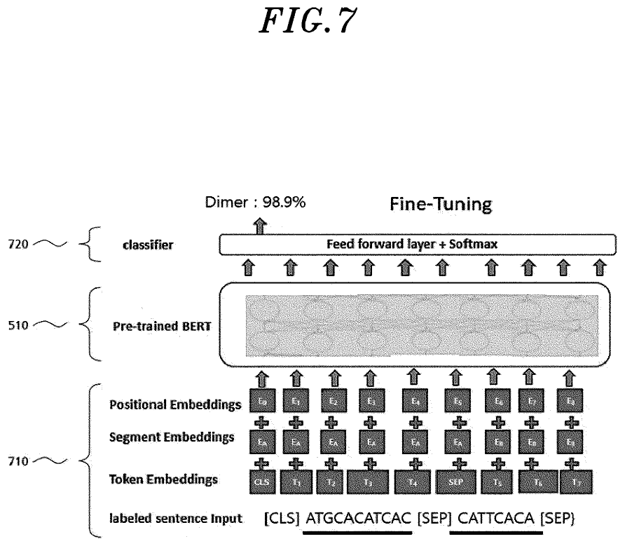

The above-described training may be performed using each of the plurality of training data sets. More detailed descriptions thereof are provided below with reference to FIG. 7.

FIG. 7 is a view illustrating a structure and an operation of a BERT-based dimer prediction model in the fine-tuning process according to an embodiment.

Referring to FIG. 7, the dimer prediction model may include BERT. In this example, the BERT may refer to a model in which supervised learning-based fine-tuning is applied to the pre-trained model 510 pre-trained using semi-supervised learning. In an embodiment, the dimer prediction model may include derivative models of BERT such as ALBERT, ROBERTa, and ELECTRA.

In an embodiment, the dimer prediction model may include at least a part of layers of the pre-trained model 510. For example, the dimer prediction model may include the input embedding layer 310 and the encoder layer 320 among layer of the pre-trained model 510 for which weights have already been calculated.

In an embodiment, the dimer prediction model may include at least one of an input embedding layer 710, the pre-trained model 510 and a second classifier layer 720.

In an embodiment, the input embedding layer 710 may convert the sequence data of oligonucleotides, which are a series of input data, into a form computable by an encoder. Depending on the embodiment, the input embedding layer 710 may correspond to the input embedding layer 310 of the pre-trained model 510.

In an embodiment, the input embedding layer 710 may include at least one of a token embedding layer for tokenizing bases of the first sequence (e.g., the forward sequence) and the second sequence (e.g., the reverse sequence) in the sequence data of oligonucleotides, a segment embedding layer for differentiating between the first sequence and the second sequence, and a position (or positional) embedding layer for applying a position data to vectors.

In an embodiment, the token embedding layer may join sequences of the two or more oligonucleotides in the training input data by using a discrimination token. For example, using a SEP (Special Separator) token which is a special token for discrimination of the first sequence and the second sequence, the token embedding layer may join the first sequence and the second sequence. Specifically, the token embedding layer may insert a first SEP token into the last position of the first sequence, insert a second SEP token into the last position of the second sequence, and link the first sequence and of the second sequence. In addition, the token embedding layer may insert a CLS (Special Classification) token for discrimination of a start position of an entire input, into the very first position of the joined sequences. Accordingly, the joined sequences may refer to a data in which the CLS token, the first sequence, the first SEP token, the second sequence and the second SEP token are linked in order.

In an embodiment, the token embedding layer may tokenize the joined sequences to obtain a plurality of tokens. For example, as described above, the token embedding layer may tokenize by dividing the joined sequences using the k-mer technique, or output by slicing the joined sequences using gene prediction technique with a function unit. Further, the token embedding layer may process each token into a vector.

In an embodiment, the segment embedding layer may process the plurality of tokens so that segment data for discriminating the sequences of two or more oligonucleotides is applied to the plurality of tokens. For example, the segment embedding layer may use two vectors, where the first vector of the two vectors (e.g., index 0) may be assigned to all tokens belonging to the first sequence, and the last vector of the two vectors (e.g., index 1) may be assigned to all tokens belonging to the second sequence.

In an embodiment, the position (or positional) embedding layer may apply a position data to the embedding vectors generated from the plurality of tokens through the token embedding and the segment embedding before the embedding vectors are used as an input of an encoder.

Embedded data processed into a form computable by an encoder while passing through the above-described input embedding layer 710 may be provided as an input to the pre-trained model 510 (e.g., pre-trained BERT). Accordingly, a context vector in which the input embedded data are comprehensively considered may be output from the pre-trained model 510.

In an embodiment, the second classifier layer 720 may predict as to occurrence and/or non-occurrence of dimer using the result output from the pre-trained model 510. For example, the second classifier layer 720 may include a classifier performing a classification function, wherein the classification function is for predicting the dimerization probability value 540 using an embedding vector (e.g., a context vector) output from a last encoder block of the pre-trained model 510 as an input. As an example, the second classifier layer 720 may output the dimerization probability value 540 for the class corresponding to ‘occurrence of dimer’ through the classifier. Alternatively, the second classifier layer 720 may output a probability value of each of a first class corresponding to ‘occurrence of dimer’ and a second class corresponding to ‘non-occurrence of dimer’. Alternatively, the second classifier layer 720 may output a classification result including a class whose probability value is (i) larger among probability values of each of the first class and the second class or (ii) larger than a preset reference value.

In an embodiment, the second classifier layer 720 may include a fully connected (FC) neural network and a SoftMax function for dimer prediction, and may be configured to perform a classification function for outputting a result of the dimerization probability value 540. For example, all embedding vectors output from the pre-trained model 510 may be input to a feed forward neural network having an FC structure, and the SoftMax function may be used as an activation function in an output layer of the feed forward neural network. A vector of a specific dimension output from the neural network may be converted into a vector with a real value between 0 and 1 with a total sum of 1 by passing the SoftMax function, and the vector may be output as the dimerization probability value 540.

Meanwhile, FIG. 7 illustrates an example of obtaining an output of the prediction result by preprocessing and embedding two sequences in the sequence data of oligonucleotides which are training input data to predict a dimerization between the two sequences, but the present disclosure is not limited to thereto. Depending on the embodiment, three or more sequences in the sequence data of oligonucleotides, which are training input data, may be used to predict a dimerization within the three or more sequences. An embodiment of the present disclosure using the three or more sequences may be performed in a manner similar to the embodiments described above or modified. For example, the sequence data of oligonucleotides may include several different pair sequences containing a forward sequence and a reverse sequence each, and the input embedding layer 710 may join all the several different pair sequences by using the discrimination token and tokenize them to be input to the pre-trained model 510.

The dimer prediction model may be obtained through the process of the fine-tuning described above. In this way, the dimer prediction model may be learned to predict a dimerization of oligonucleotides by fine-tuning the pre-trained model 510 pre-trained as to a pattern of nucleic acid sequences.

In an embodiment, a data of a reaction condition may be further used in the process of the fine-tuning. Here, the reaction condition may broadly refer to a reaction environment of a nucleic acid amplification reaction, a condition for materials added for the reaction environment, or and so on.

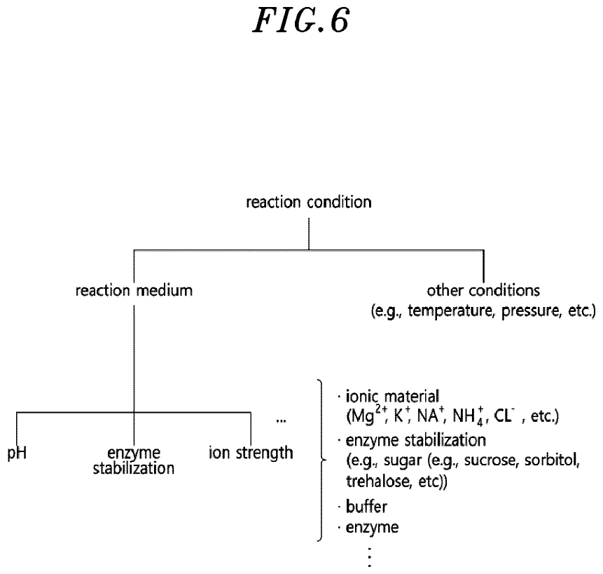

FIG. 6 is a view illustrating a concept of reaction conditions according to an embodiment.

Referring to FIG. 6, the reaction condition may comprise at least one of a reaction medium used in the nucleic acid amplification reaction and other conditions.

Here, the reaction medium refers to a material surrounding a reaction environment. In an embodiment, the reaction medium may include materials added for providing a favorable reaction environment in at least one of a plurality of steps in a nucleic acid amplification reaction. The plurality of steps in the nucleic acid amplification reaction may include, for example, denaturing step, annealing step, and extension (or amplification) step for amplifying a DNA (deoxyribonucleic acid) having a target nucleic acid sequence in a reaction well containing a sample comprising the target nucleic acid sequence. Meanwhile, in an embodiment, the reaction medium may be broadly interpreted to encompass other conditions described below.

In an embodiment, the reaction medium may comprise at least one selected from the group consisting of a pH-related material, an ion strength-related material, an enzyme, and an enzyme stabilization-related material. For example, multiple groups of the reaction medium may be designed with differences in at least one of pH-related material, the ion strength-related material, the enzyme and the enzyme stabilization-related in terms of their types and/or concentrations. In addition, detailed conditions of the reaction medium may be defined after any one of the plurality of groups is selected by a user. Since the reaction medium affects pH, ionic strength, activation energy, and enzyme stabilization in the reaction well containing oligonucleotides, a difference in the reaction medium may cause a difference in a dimerization of oligonucleotides.

In an embodiment, the pH-related material may be a material affecting pH in the process of the nucleic acid amplification reaction or a material added to a reaction well for giving a specific pH value or range. For example, the pH-related material may comprise a buffer. As an example, the buffer may comprise Tris buffer and EDTA (ethylene-diamine-tetraacetic acid).

In an embodiment, the ion strength-related material may be a material affecting ion strength in the process of the nucleic acid amplification reaction or a material added to a reaction well for giving a specific ion strength. For example, the ion strength-related material may comprise an ionic material. As an example, the ionic material may comprise Mg²⁺, K⁺, Na⁺, NH⁴⁺, and Cl⁻, etc. In an embodiment, an oligonucleotide may refer to the ion strength-related material because it contains anions.

In an embodiment, the enzyme stabilization-related material may be a material added to a reaction well for enzyme stabilization in the process of the nucleic acid amplification reaction. For example, the enzyme stabilization-related material may comprise a sugar. As an example, the sugar may comprise a sucrose, a sorbitol, and trehalose, etc.

In an embodiment, the enzyme may comprise an enzyme involved in the process of the nucleic acid amplification reaction. For example, the enzyme may comprise a nuclease used for cleaving a nucleic acid molecule (e.g., DNA exonuclease, DNA endonuclease, RNase), a polymerase used in a polymerization reaction of a nucleic acid molecule (e.g., DNA polymerase, reverse transcriptase, terminal transferase), a ligase used for linking a nucleic acid molecule, and a modifying enzyme used for adding or removing various functional groups (e.g., Uracil-DNA glycosylase). As the above-described enzyme, various types of enzymes known in the field of molecular diagnostics may be used, and the enzyme is not limited to the above-described examples. In an embodiment, the enzyme may comprise Taq DNA polymerase with 5′ to 3′ exonuclease activity, reverse transcriptase and Uracil-DNA glycosylase.

In addition, other conditions may comprise a factor such as temperature, pressure, and time, etc. In an embodiment, other conditions may comprise a condition such as a reaction temperature, a reaction pressure, and/or a reaction time applied to a reaction well for at least one of several steps for the nucleic acid amplification reaction described above. For example, other conditions may include a reaction temperature or a reaction time during the extension step in which an oligonucleotide is bound to a target nucleic acid and extended.

In addition, the reaction condition may be determined in consideration of the reaction medium and other conditions described above. In an embodiment, a multiple group of the reaction condition may be designed by varying the types, concentrations or magnitudes of at least one of the reaction mediums and other conditions. For example, the types or concentrations of at least one of the pH-related material, the ion strength-related material, the enzyme, and the enzyme stabilization-related material may be differently determined for each reaction condition.

Referring to embodiments for the reaction condition described above, the dimer prediction model may be obtained by further considering the reaction condition described above. Specifically, the dimer prediction model may be obtained according to the following embodiments.

In a first embodiment, the dimer prediction model may be generated for each of the reaction conditions described above. Specifically, the dimer prediction model may comprise a plurality of models generated by fine-tuning the pre-trained model 510 for each of the reaction conditions. More specifically, the plurality of models corresponding to the plurality of reaction conditions are individually generated using the pre-trained model 510 as described above with reference to FIG. 7. Each model may be fine-tuned using training data sets tested under each reaction condition. Accordingly, when the sequences of the oligonucleotides are input, each model may predict whether a dimerization of the oligonucleotides occurs under the corresponding reaction condition based on training under the corresponding reaction condition.

In the first embodiment, training data sets are collected for each reaction condition (or training data sets further containing an additional data of reaction conditions may be classified according to each reaction condition), the fine-tuning of the pre-trained model 510 for each reaction condition may be performed by using a training data set for each reaction condition. In addition, the plurality of models learned to predict a dimerization under each reaction condition may be provided depending on the reaction conditions. In an example, the plurality of models may be generated by connecting a classifier to an output of each of the plurality of pre-trained model 510 and then each of the plurality of models may be fine-tuned using training data sets for each reaction condition.

In the first embodiment, training data sets in which settings for reaction conditions are at least partially the same may be collected or sorted. For example, for reaction conditions including reaction medium, reaction temperature and reaction time, a first reaction condition with a first set of settings, a second reaction condition with a second set of settings, and a N^{th} (N is a natural number not less than 2) reaction condition with a N^{th} set of settings may be determined, training data sets having reaction condition corresponding to each of the first to N^{th} reaction condition may be sorted from pre-stored training data sets. Further, a first model corresponding to the first reaction condition, a second model corresponding to the second reaction condition, and a N^{th} model corresponding to the N^{th} reaction condition may be obtained through fine-tuning using training data sets corresponding to each of the first to N^{th} reaction condition.

In a second embodiment, the dimer prediction model may be learned to predict a dimerization using both a data of reaction conditions and the sequence of the oligonucleotides. Specifically, the training input data may further comprise a data of the reaction condition, and the dimer prediction model may comprise one model generated by fine-tuning the pre-trained model 510 using the plurality of training data sets. For example, the training input data used in the fine-tuning may comprise the sequence data of oligonucleotides and the data of the reaction condition, and the training answer data may comprise a label data as to occurrence and/or non-occurrence of dimer of the oligonucleotides obtained from a result performed under the corresponding reaction condition. The dimer prediction model as one model may predict a dimerization using both the data of reaction conditions and the sequence of the oligonucleotides and the dimer prediction model considering the reaction conditions may be trained by comparing the predicted result with the training answer data.

In the second embodiment, the training data sets further comprising the data of reaction conditions as the training input data may be collected and then the fine-tuning of the pre-trained model 510 using the training data sets may be performed. In an example, all data in the training input data may be processed as the embodiment described above to be used as inputs of the dimer prediction model. Alternatively, the data of reaction conditions in the training input data may be provided to one or more neural networks or layers to be used as an input. Further, where both a data of the reaction conditions and a sequence of the oligonucleotides are input to the dimer prediction model in the fine-tuning, the dimer prediction model learned to predict a dimerization of oligonucleotides in considering reaction conditions may be provided.

Table 2 illustratively shows the plurality of training data sets used in the fine-tuning according to an embodiment. As shown in Table 2, each training data set may comprise inputs as to the first sequence (e.g., the forward sequence), the second sequence (e.g., the reverse sequence) and the reaction condition for the experiment (e.g., identifier of the reaction condition), and a label as to whether a dimerization between the first sequence and the second sequence occurs or not under the corresponding reaction condition (e.g., a label ‘1’ corresponds to ‘occurrence of a dimerization’ and a label ‘0’ corresponds to ‘non-occurrence of a dimerization’). In Table 2, M as a natural number may be less than N, and the reaction condition for individual samples may be same or different from each other.

TABLE 2

| | | | Reaction | |
|---|---|---|---|---|
| | First sequence | Second sequence | condition | Label |
| Set1 | AGCATTGTGGGTAGTAAGGTATAAA | AGCTCAAAATCTACATAACCCCTC | 1 | 1 |
| Set2 | AGCGTTATTGTTGAGAAATGGATTG | AGCACAAAAAAATTTATACAAAAAACAACT | 2 | 0 |
| ... | ... | ... | ... | ... |
| SetN-1 | AGCGTGGTTATTGGATGGGTTTG | AGCAAATCTTTACTAAAAAAAATTTACCTT | M-1 | 1 |
| SetN | AGCTGTTTTTTTTTTTGTTGTGGGTAA | AGCCTATAAATCCTAATACTTAACTCA | M | 0 |

In an embodiment, a plurality of hyper-parameters may be used in each of the pre-training and the fine-tuning described above. The hyper-parameters may refer to variables changeable by the user. The hyper-parameters may include, for example, a learning rate, a cost function, a count of learning cycle repetitions, a weight initialization (e.g., setting a range of weight values subject to weight initialization), and a count of Hidden Unit (e.g., a count of hidden layers, a count of nodes in hidden layers), etc. In addition, the hyper-parameters may further include the tokenization technique described above (e.g., the k-mer technique, the gene prediction technique), a setting value of k in the k-mer technique, and a step size (e.g., gradient accumulation step), a batch size and a dropout in learning by a gradient descent method.

In an embodiment, hyper-parameters used in the fine-tuning may be different from hyper-parameters used in the pre-training. For example, hyper-parameters used in the fine-tuning may further include a focusing parameter for resolving an answer imbalance problem (or a class imbalance problem) described below.

In an embodiment, before performing the fine-tuning, a process for determining whether there is the answer imbalance problem may be performed by analyzing the result of the dimer experiment to be used as the training data sets for the fine-tuning. Here, the answer imbalance problem refers to a case where class variables of the training data sets are not uniformly distributed but are relatively biased towards one value. When the answer imbalance problem is present, it may lead to a problem of poor prediction performance for relatively minority classes.

In an embodiment, when it is determined that the answer imbalance problem is present, a learning method weighting greater weights to cases difficult related to calculating a loss (e.g., a cross entropy loss) or easily misclassified in the process of the fine-tuning may be applied. For example, weight given to easy samples may be down-scaled by lowering the weight, and weight given to difficult samples may be up-scaled by increasing the weight.

In an embodiment, a focal loss technique may be used to solve the answer imbalance problem. For example, when it is determined that the answer imbalance problem is present, a weight scaling using Math FIGS. 1 and 2 may be performed in the process of the fine-tuning.

$$F L = - {( 1 - {P}_{t} )}^{γ} log ( {P}_{t} ) \qquad MATH FIGURE 1$$

$$CE = - log ( {P}_{t} ) \qquad MATH FIGURE 2$$

Here, CE refers to a cross entropy, FL refers to a focal loss, and γ as a focusing parameter refers to a rate at which weights of easy problems are down-weighted. In addition, (1−Pₜ)^{γ} refers to a modulating factor that allows easy samples to be down-scaled to focus on difficult samples. For example, when a specific sample of the training data sets is misclassified and Pₜ is small, the modulating factor (1−Pₜ)^{γ} is close to 1, so the loss may not be affected. However, on the other hand, the better the sample is classified, and the closer Pₜ is to 1, the closer the modulating factor is to 0 and the loss may be down-weighted.

According to an embodiment of the present disclosure, in order to derive optimized hyper-parameters in the process of the fine-tuning, a method of dividing the plurality of the training data sets, performing a plurality of training under different conditions using the divided training data sets, and using the training results may be used.

Specifically, the plurality of the training data sets may be grouped into p groups (p is a natural number) in the process of the fine-tuning. Here, each group refers to a training data sets group including training data sets, each arranged as a unit of a pair of training input data and training answer data. For example, if a count of total training data sets is 1,000, the training data sets may be divided into 5 groups having 200 training data sets each.

In an embodiment, the dimer prediction model may be trained using some of the p groups, and a performance verification of the dimer prediction model may be performed using the remaining groups excluding the some of the p groups. For example, the total training data sets may be divided into 5 groups, and each of four pre-trained models 510 to which different hyper-parameter values are applied may be trained using the training data sets in each of 4 groups from the 5 groups. Accordingly, four different exemplary dimer prediction models may be generated as targets for the performance verification. Further, the performance verification of the above four different exemplary dimer prediction models may be performed using the training data sets in the remaining one group except for the 4 groups from the 5 groups.

In an embodiment, hyper-parameter values applied to training the dimer prediction model may be updated based on results of the performance verification. For example, by comparing the results of the performance verification of the four different exemplary dimer prediction models described above, any one exemplary dimer prediction model with a best evaluation score may be selected. Further, hyper-parameter values applied to the selected exemplary dimer prediction model may be determined as hyper-parameter values to be used in the process of the fine-tuning.

In an embodiment, a K-fold cross validation technique may be used in a process of determining hyper-parameter values. For example, among the total training data sets that can be used for the fine-tuning, the remaining training sets except for test sets may be divided into K equal parts (e.g., K=5, 10). Among them, 1/K may be used as validation sets and (K−1)/K may be used as training sets. This process may be repeated K times for each divided data sets. As a result, K models may be generated and a MSE (Mean Squared Error) value of the dimer prediction model may be determined based on an average of the MSE value of each model.

Table 3 shows performance verification scores of the dimer prediction model according to an embodiment. A first method to a third method are conventional techniques. The first method is a conventional pattern law-based prediction calculation method, the second method is a method using a conventional Nupack algorithm, and the third method is a method without pre-training. Further, each of Example 1 to Example 4 refers to the dimer prediction model which is transfer learned by using the training data of each of Sars-Cov2, Corona, RNA virus, and viruses, based on a 3-mer tokenization technique. Table 3 shows various verification scores of the first method to the third method and Example 1 to Example 4, such as an accuracy, a precision, a recall, a F1 score (a harmonic mean of the precision and the accuracy), and an AUROC (area under ROC, measurement graphs of the model's classification performance at various thresholds). Table 3 shows that the examples of the present disclosure secured a significantly high evaluation score when comparing with the conventional techniques.

When comparing the verification scores, it may be seen that the examples of the present disclosure secured a significantly high evaluation score.

TABLE 3

| | First | Second | Third | | | | |
|---|---|---|---|---|---|---|---|
| | method | method | method | Example 1 | Example 2 | Example 3 | Example 4 |
| Accuracy | 0.6795 | 0.7116 | 0.6040 | 0.7122 | 0.7571 | 0.7088 | 0.7119 |
| Precision | 0 | 0.4552 | 0.4471 | 0.4635 | 0.5170 | 0.4431 | 0.5231 |
| Recall | 0 | 0.7625 | 0.5423 | 0.7692 | 0.7026 | 0.7679 | 0.8308 |
| F1 | 0 | 0.5701 | 0.3819 | 0.5657 | 0.5841 | 0.5577 | 0.5875 |
| AUROC | 0.5 | 0.7230 | 0.5326 | 0.7125 | 0.7225 | 0.7290 | 0.7702 |

Meanwhile, the fine-tuning according to an embodiment may comprise (i) a first fine-tuning the pre-trained model 510 using a plurality of a first training data sets for sequences of two oligonucleotides, and (ii) a second fine-tuning the model obtained as a result of the first fine-tuning using a plurality of a second training data sets for sequences of three or more oligonucleotides.

In an embodiment, the training input data of each of the first training data sets may comprise a sequence data of one pair of oligonucleotides comprising a forward sequence and a reverse sequence, and the training answer data of each of the first training data sets may comprise a label data which is determined from a result of a dimer experiment (e.g., whether a dimerization occurs) as to a nucleic acid amplification reaction performed in a singleplex environment, wherein the singleplex environment refers that the one pair of oligonucleotides are contained in one tube. In an embodiment, the pre-trained model 510 may be fine-tuned at first by using the plurality of the first training data sets. As a result of the first fine-tuning, the dimer prediction model learned to predict a dimerization of one pair of oligonucleotides when sequences of the one pair of oligonucleotides are input may be obtained.

In an embodiment, the training input data of each of the second training data sets may comprise a sequence data of multiple pair of oligonucleotides comprising a forward sequence and a reverse sequence each, and the training answer data of each of the second training data sets may comprise a label data which is determined from a result of a dimer experiment (e.g., whether a dimerization is occurs) as to a nucleic acid amplification reaction performed in a multiplex environment, wherein the multiplex environment refers that the multiple pair of oligonucleotides are contained in one tube. In an embodiment, the dimer prediction model fine-tuned using the plurality of a first training data sets may be further fine-tuned using the plurality of a second training data sets. As a result of the second fine-tuning, the dimer prediction model learned to predict a dimerization of multiple pair of oligonucleotides in the multiplex environment even if sequences of multiple pair of oligonucleotides are input, may be obtained. Accordingly, the dimer prediction model capable of more accurately predicting a dimerization when multiple oligonucleotide pairs are contained in the multiplex environment may be implemented.

As described above, the dimer prediction model may be obtained through the pre-training and the fine-tuning. As described below, the dimer prediction model may be transfer-learned to output a prediction result for a dimerization of one or more oligonucleotides when a sequence data of the one or more oligonucleotides for which the dimerization is to be predict is input to the dimer prediction model. The computer device 100 may store and manage the dimer prediction model and provide the dimer prediction model. For example, the computer device 100 may be implemented so that the server stores and manages the dimer prediction model learned by the transfer learning method and the server provides the dimer prediction model to the user terminal when the user terminal requests the dimer prediction model.

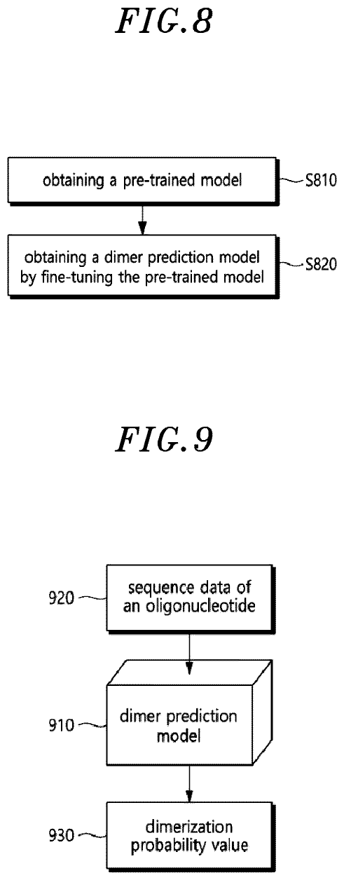

FIG. 8 is an exemplary flowchart for obtaining a dimer prediction model according to an embodiment.

In an embodiment, steps shown in FIG. 8 may be performed by the computer device 100. In a further embodiment, the steps shown in FIG. 8 may be implemented by a single entity, such as the steps are performed in the server. In another embodiment, the steps shown in FIG. 8 may be implemented by a plurality of entities, such as some of the steps are performed in the user terminal and others are performed in the server.

In Step S810, the computer device 100 may obtain the pre-trained model 510. In an embodiment, the pre-trained model 510 may use the plurality of nucleic acid sequences 220 as the training data. As an example, the computer device 100 may obtain the plurality of nucleic acid sequences 220 and obtain the pre-trained model 510 by training the language model 510 using the training data comprising the plurality of nucleic acid sequences 220. As another example, the computer device 100 may receive the pre-trained model 510 that has already been learned by another device, from that another device or the storage medium (e.g., database, etc.).

In an embodiment, the plurality of nucleic acid sequences 220 may be obtained from a specific group of an organism. In an embodiment, the pre-trained model 510 may be trained by the semi-supervised learning method performed in which a mask to some of bases in the nucleic acid sequences is applied and then an answer of the masked base is found. In an embodiment, the pre-trained model 510 may be trained by using nucleic acid sequences tokenized with tokens each having two or more bases. In an embodiment, the tokens may comprise each bases tokenized by (i) dividing the nucleic acid sequences by k unit or (ii) dividing the nucleic acid sequences by a function unit.

For example, the computer device 100 may collect large amounts of sequences of target analytes form public databases such as NCBI, and GISAID, etc. The computer device 100 may obtain the pre-trained model 510 by pre-training sequences without labeling using the MLM method, which applies a mask to some of a sequence and then find an answer of the masked some of the sequence, using the BERT language model.

In Step S820, the computer device 100 may obtain the dimer prediction model 910 by fine-tuning the pre-trained model 510. For example, the computer device 100 may obtain a plurality of training data sets and perform fine-tuning on the pre-trained model 510 using the plurality of training data sets.

In an embodiment, the fine-tuning may be performed using the plurality of training data sets, each training data set comprises (i) the training input data comprising a sequence data of two or more oligonucleotides and (ii) the training answer data comprising a label data as to occurrence and/or non-occurrence of dimer of the two or more oligonucleotides. In an embodiment, the fine-tuning may comprise (i) joining sequences of the two or more oligonucleotides by using the discrimination token and (ii) tokenizing the joined sequences to obtain a plurality of tokens.

For example, the computer device 100 may receive a plurality of dimer experiment results from another device or the storage medium, each dimer experiment result including (i) sequences of oligonucleotides (e.g., forward sequence, reverse sequence) and (ii) data related to occurrence and/or non-occurrence of dimer in the experiment. Further, the computer device 100 may generate the plurality of training data sets described above from the results of the dimer experiment. In addition, the computer device 100 may determine the structure of the dimer prediction model by adding the layer for dimer prediction 520 comprising a FC feed forward neural network and a SoftMax function to the pre-trained model 510, and may train the dimer prediction model to predict a dimerization probability value by using the training data sets. As an example, the computer device 100 may input the forward sequence and the reverse sequence in each training data set into the model in a form of vectors that can be calculated by the encoders of the dimer prediction model, based on a method in which the sequences are joined by using the [SEP] token and then the joined sequences are tokenized and embedded using the k-mer method.

Various embodiments of the process performed in which the dimer prediction model is learned by the transfer learning method are described above. As seen above, the computer device 100 may obtain the dimer prediction model through the pre-training for sequences of target analytes and the fine-tuning for dimer prediction, based on a fact that sequences of oligonucleotides used in diagnostic reagents are part of sequences of target analytes (e.g., virus). Accordingly, high prediction performance may be achieved even when using a relatively small amount of labeled data.

Various embodiments of a process for predicting a dimerization using the dimer prediction model presented above are described below.

The computer device 100 may provide an input data comprising a sequence data of one or more oligonucleotides to the dimer prediction model learned by the transfer learning method and obtain a prediction result for a dimerization of the one or more oligonucleotides from the dimer prediction model. In an embodiment, the computer device 100 may control the dimer prediction model to be executed and display the prediction result for the dimerization of the one or more oligonucleotides output from the dimer prediction model as the sequence data of the one or more oligonucleotides is input into the dimer prediction model.

FIG. 9 is a view illustrating a concept of an inference operation by the dimer prediction model 910 according to an embodiment. In an embodiment, the dimer prediction model 910 shown in FIG. 9 may refer to a model on which the fine-tuning or the transfer learning of the pre-trained model 510 has been completed.

Referring to FIG. 9, the dimer prediction model 910 may output a dimerization probability value 930 when a sequence data 920 of an oligonucleotide is input. Here, the oligonucleotide refers to a target for dimer prediction and refers to one or more oligonucleotides to be predicted as to occurrence and/or non-occurrence of dimer of the oligonucleotide using a result of the transfer learning.

In an embodiment, the sequence data 920 of the oligonucleotide may include a forward sequence and a reverse sequence of the oligonucleotide. This embodiment may be interpreted in a similar way to the above-described embodiments related to process of the fine-tuning. In an embodiment, the sequence data 920 of the oligonucleotide may be obtained based on a user input for the sequence of the oligonucleotide, loaded from the memory 110, or received from another device (e.g., storage medium, etc.).

In an embodiment, when the sequence data 920 of the oligonucleotide is input, the dimer prediction model 910 may join sequences of one or more oligonucleotides in the sequence data 920 of the oligonucleotide by using a discrimination token, and tokenize the joined sequences to obtain a plurality of tokens. The dimer prediction model 910 may generate a context vector from the plurality of tokens and predict the dimerization probability value 930 using the context vector.

A structure and an operation of the dimer prediction model 910 for predicting the dimerization probability value 930 may be interpreted in a similar way to the embodiments that the dimer prediction model outputs the dimerization probability value 540 during the process of the fine-tuning shown in FIG. 5 or FIG. 7.

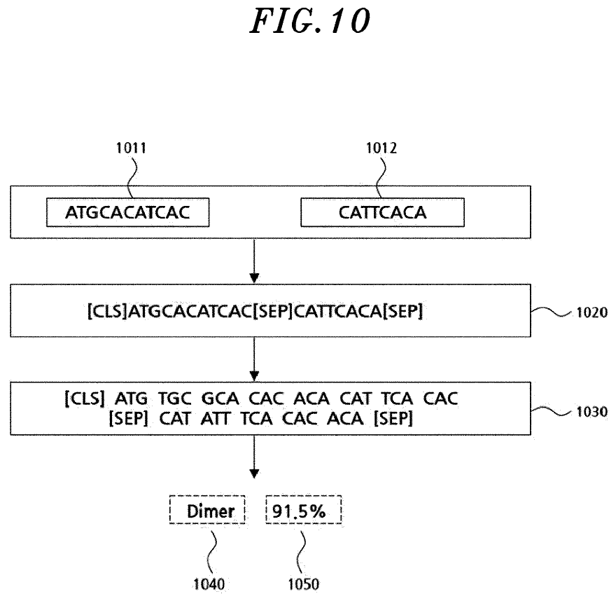

FIG. 10 is a view illustrating an example method for predicting the dimerization probability 930 by the dimer prediction model according to an embodiment.

Referring to FIG. 10, the dimer prediction model 910 may obtain the sequence data 920 of the oligonucleotide including a sequence 1011 of the first oligonucleotide and a sequence 1012 of the second oligonucleotide.

In an embodiment, the dimer prediction model 910 may join the sequence 1011 of the first oligonucleotide and the sequence 1012 of the second oligonucleotide by using the discrimination token. For example, as described above, the dimer prediction model 910 may obtain the joined sequences 1020, by inserting the SEP token for sequence discrimination into the last position each of the first sequence and the second sequence and inserting the CLS token for start position discrimination into the very first position of the entire input.

In an embodiment, the dimer prediction model 910 may obtain a plurality of tokens 1030 corresponding to the joined sequences 1020. For example, at least one token may be generated for each of the bases included in the joined sequences 1020 joined with the SEP token. As an example, the dimer prediction model 910 may receive the sequence data 920 of the oligonucleotide and generate the plurality of tokens 1030 by performing preprocessing the input sequence data 920 of the oligonucleotide, as described above. As another example, the dimer prediction model 910 may receive the plurality of tokens 1030 as the sequence data 920 of the oligonucleotide.

In an embodiment, each of the plurality of tokens 1030 may comprise a plurality of bases. At least a part of the sequence 1011 of the first oligonucleotide or the sequence 1012 of the second oligonucleotide in tokens adjacent to each other among the plurality of tokens 1030 may overlap. For example, the plurality of tokens 1030 from the joined sequences 1020 may be generated so that a first token comprises ATG, a second token adjacent to the first token comprises TGC, and a third token adjacent to the second token comprises GCA. As in the example above, the first token and the second token may comprise bases T and G in different positions, and the second token and third token may comprise bases G and C in different positions. In this way, tokens may be generated in a method in which common bases of adjacent tokens overlap.

In an embodiment, the dimer prediction model 910 may output one or more prediction value 1050 corresponding to one or more class 1040 of the dimer prediction model 910, using the plurality of tokens 1030. For example, the dimer prediction model 910 may generate prediction values 1050 (e.g., the dimerization probability value 930) corresponding to pre-determined classes (e.g., occurrence of dimer, non-occurrence of dimer) each. In an embodiment, a type or a count of classes of the dimer prediction model 910 may be variably determined depending on the implementation aspect.

In an embodiment, the process shown in FIG. 10 may at least partially correspond to the process for predicting the classification value or the probability value as the result of prediction by the dimer prediction model in the above-described fine-tuning.

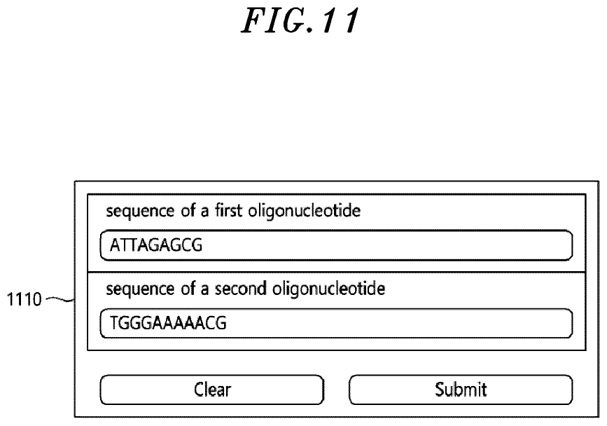

FIG. 11 is a view illustrating an example process in which the sequence data 920 of the oligonucleotide is obtained through a user input according to an embodiment.

Referring to FIG. 11, the computer device 100 according to an embodiment may obtain the sequence data 920 of the oligonucleotide based on a user input. For example, the computer device 100 may display a user interface screen 1110 for requesting input of oligonucleotide sequences. Further, the computer device 100 may receive user input for sequences of oligonucleotides pairing a sequence of a first oligonucleotide (e.g., the forward primer sequence) and a sequence of a second oligonucleotide (e.g., the reverse primer sequence) through the user interface screen 1110. In another embodiment, the computer device 100 may receive the sequence data 920 of the oligonucleotide from a storage medium, etc., connected the computer device 100.

In an embodiment, a user input for the reaction conditions may be received in a method similar to the example in FIG. 11, For example, identification numbers of the reaction conditions may be entered by a user, one of a plurality of lists for the reaction conditions may be selected by the user, or a plurality of lists of the reaction mediums within the reaction conditions may be selected by the user.

The computer device 100 may obtain a prediction result for the dimerization of the oligonucleotide from the dimer prediction model 910. In an embodiment, the computer device 100 may obtain the prediction result based on the output of the dimer prediction model 910 (e.g., the dimerization probability value 930). For example, the computer device 100 may obtain the prediction result including the output (e.g., the dimerization probability value 930 or the classification result for the dimerization (e.g., ‘occurrence of dimer’ or ‘non-occurrence of dimer’)) of the dimer prediction model 910 or a post-processed result (e.g., additional calculations, unit adjustments, other modification, etc.) from the output.

The computer device 100 may obtain the prediction result comprising one of a plurality of preset prediction classifications based on whether the dimerization probability value 930 belongs to any one of a plurality of preset ranges, when the dimerization probability value 930 is obtained from the dimer prediction model 910. For example, the plurality of prediction classifications may comprise high level, medium level, and low level of dimerization probability, and each prediction classification may correspond to a different range of probability values.

The computer device 100 according to an embodiment of the present disclosure may obtain the prediction result for the dimerization of the oligonucleotide by considering the above-described reaction conditions. Specifically, the prediction result may be obtained according to the following embodiments.

As described above as to the fine-tuning, the dimer prediction model 910 according to the first embodiment may be generated in accordance with each of reaction conditions. That is, the dimer prediction model 910 may comprise a plurality of models (e.g., the first model to the Nth model) generated by fine-tuning each of a plurality of the pre-trained model 510 in accordance with each of a plurality of the reaction conditions (e.g., the first reaction condition to the Nth reaction condition). The computer device may control at least some of the plurality of models to be executed.

In the first embodiment, the computer device 100 may obtain a plurality of prediction results for the dimerization from the plurality of models. For example, the computer device 100 may input the sequence data 920 of the oligonucleotide into each of the first model to the Nth model corresponding to the first reaction condition to the Nth reaction condition. The computer device 100 may obtain the dimerization probability value 930 from each of the first model to the Nth model. The computer device 100 may output the dimerization probability values 930 of each of the first model to the Nth model and a description (e.g., enzyme master mix info., reaction condition identifier) of each reaction condition corresponding each model, as the plurality of prediction results.

In the first embodiment, the computer device 100 may obtain a prediction result from a model corresponding to a reaction condition matched to the input data among the plurality of models. For example, the computer device 100 may also obtain the data of the reaction condition when obtaining the sequence data 920 of the oligonucleotide, and match the obtained data of the reaction condition to the corresponding sequence data 920 of the oligonucleotide. Further, the computer device 100 may input the sequence data 920 of the oligonucleotide into one or more model corresponding to the reaction condition matching the sequence data 920 of the oligonucleotide among the first model to the Nth model. The computer device 100 may output the dimerization probability values 930 as the prediction result, by obtaining the dimerization probability value 930 from the corresponding model. Alternatively, the computer device 100 may input the sequence data 920 of the oligonucleotide into each of the first model to the Nth model and obtain the dimerization probability value 930 from each of the first model to the Nth model. The computer device 100 may output a result in which an identifiable indicator (e.g., highlight) is added to one or more corresponding to the reaction condition matching the sequence data 920 of the oligonucleotide among of these N dimerization probability values 930 as the prediction result, or the computer device 100 may output a result in which the N dimerization probability values are calculated using a pre-determined method (e.g., average) as the prediction result.

According to the second embodiment, the dimer prediction model 910 may be one model generated by fine-tuning the pre-trained model 510 using the plurality of the training data sets further comprising the data of the reaction condition. The dimer prediction model 910 may be learned to predict a dimerization when both the sequence of the oligonucleotide and the data of the reaction condition are input.

In the second embodiment, the input data to be provided to the dimer prediction model 910 may further comprise a data of a reaction condition, whereby the prediction result for the dimerization may be obtained based on the sequence data and the data of the reaction condition. For example, the computer device 100 may also obtain the data of the reaction condition when obtaining the sequence data 920 of the oligonucleotide, and input the input data comprising the sequence data 920 of the oligonucleotide and the data of the reaction condition into the dimer prediction model 910. The computer device 100 may obtain the dimerization probability value 930, which is output from the dimer prediction model 910 based on the sequence data and the data of the reaction condition. The computer device 100 may output the obtained the dimerization probability value 930 and a description (e.g., enzyme master mix info., reaction condition identifier) of the corresponding reaction condition as the plurality of prediction results.

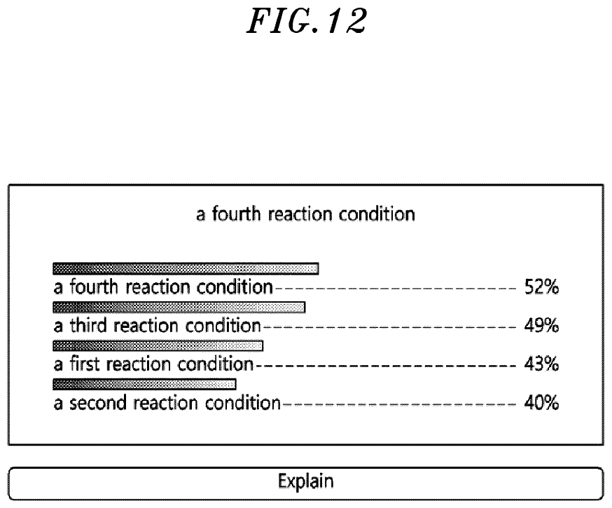

FIG. 12 is a view illustrating an example process in which a prediction result for the dimerization is output according to the first embodiment.

A result screen 1210 shown in FIG. 12 is used for illustrative purposes, and a type of graph, and scale of graph, etc., may be vary depending on the implementation mode.

Referring to FIG. 12, the computer device 100 may provide prediction results for a dimerization under a plurality of the reaction conditions. In the first embodiment in which a plurality of the dimer prediction model 910 are generated in consideration of the reaction conditions, as the sequence data 920 of the oligonucleotide is input to each of the plurality of the dimer prediction model 910, the result screen 1210 including the dimerization probability values 930 output from each of the plurality of the dimer prediction model 910 may be displayed.

For example, by using the above-described transfer learning method, the first to the fourth dimer prediction models corresponding to the first to the fourth reaction condition having different types and concentrations of enzymes in the reaction medium may be implemented. The computer device 100 may input a sequence set comprising a first forward primer sequence and a reverse primer sequence. Further, the computer device 100 may obtain a first dimerization probability value to a fourth dimerization probability value, such as, 52%, 49%, 43% and 40%, from the first dimer prediction model to the fourth dimer prediction model. The computer device 100 may display the result screen 1210, which shows (i) names or identification data of the first to the fourth reaction condition and (ii) the first dimerization probability value to the fourth dimerization probability value, as shown in FIG. 12, in a form of a comparison graph.

In an embodiment, when a mouse cursor is positioned on any one of the plurality of the reaction conditions displayed on the result screen 1210, a configuration specification data of the corresponding any one reaction condition may be overlaid on the result screen 1210.

According to an embodiment of the present disclosure, the computer device 100 may output a prediction supporting data used as a basis for prediction of the dimerization. For example, the computer device 100 may display the result screen 1210 and a basis request button 1220 together, and when a user selection input for the basis request button 1220 is received, obtain and display a prediction supporting data for each dimerization probability value 930.

In an embodiment, the prediction supporting data may be generated by using an explainable artificial intelligence (XAI). For example, by using a method (e.g., SmoothGrad, etc.) for extracting explainable features from the dimer prediction model 910 implemented as an explainable deep learning model, the computer device 100 may provide explanatory data about the extracted features through a preset explanation interface. Depending on the embodiment, other methods (e.g., LRP, etc.) for checking characteristics, weights, and main object locations of input data depending on the model may be used. For another example, the computer device 100 may obtain the prediction supporting data by using the attention algorithm used in the dimer prediction model 910. For example, the prediction supporting data may be generated by using a similarity calculated according to the attention algorithm, a key and a value reflected the similarity, and an attention value, etc. In addition, a BERT internal structural analysis algorithm (see ACL 2019) may be used in the obtaining the prediction supporting data.

Meanwhile, depending on the embodiment, a model-independent explanation method (e.g., LIME) for performing a cause analysis by checking an output obtained by adjusting an input without being dependent on the model in the obtaining the prediction supporting data may be used.

In an embodiment, the prediction supporting data may refer to a data able to be presented as the basis for prediction of the dimerization. For example, the computer device 100 may generate an analysis result for whether the prediction result for the dimerization output from the dimer prediction model 910 satisfies a plurality of pre-stored pattern rules corresponding to the dimerization, and provide the analysis result as the prediction supporting data.

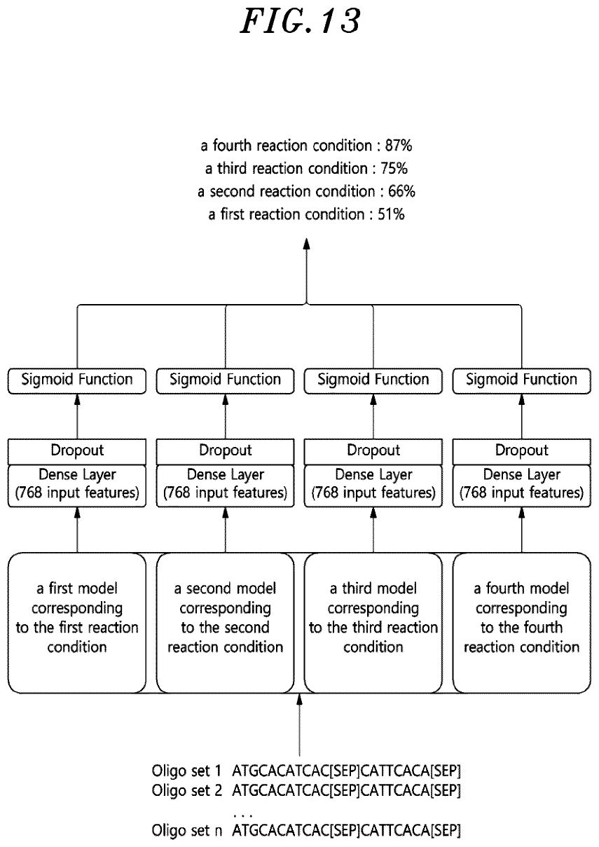

FIG. 13 is a view illustrating an example process in which the dimer prediction is performed using a plurality of oligonucleotide sequence sets of according to an embodiment.

Referring to FIG. 13, the dimer prediction using plurality of oligonucleotide sequence sets used for detecting a plurality of target analytes may be performed.

For example, the plurality of oligonucleotide sequence sets may be used to detect the plurality of target analytes. The plurality of oligonucleotide sequence sets may comprise a first oligonucleotide sequence set comprising a first forward primer sequence and a first reverse primer sequence, to a nth oligonucleotide sequence set comprising a nth forward primer sequence and a nth reverse primer sequence. As the plurality of oligonucleotide sequence sets comprising various primer pairs or probes are input into the plurality of dimer prediction models 910 considering the reaction conditions (e.g., the first model corresponding to the first reaction condition to a fourth model corresponding to a fourth reaction condition), a dimerization in the multiplex environment where the plurality of sequences are mixed in a reaction well may be predicted.

For predicting a dimerization in the above-described multiplex environment, each training data set used for the fine-tuning may comprise (i) an input comprising a plurality of oligonucleotide sequence sets different each other having a pair of the forward sequence and the reverse sequence each and (ii) a label data for a dimerization which is a result for a dimer experiment in the multiplex environment where a plurality of the pairs of oligonucleotides are contained in one tube. As in the example above, all oligonucleotide sequences in the input may be all discriminated by using the discrimination token (e.g., SEP) and provided to the model, and a training is performed using the label data that is the result for the dimer experiment on the plurality of the pairs. As the fine-tuning using the plurality of the training data sets is performed, the dimer prediction model 910 learned from results of the dimer experiment in the multiplex environment may be implemented.

For example, as described FIG. 13, each forward sequence and reverse sequence in the plurality of oligonucleotide sequence sets may be joined using [SEP] tokens, tokenized and input into each of the plurality of the dimer prediction models 910. Each dimer prediction model 910 may calculate and output the dimerization probability value 930 in the multiplex environment where the plurality of oligonucleotide sequence sets are mixed based on learning results under each reaction condition.

Depending on the embodiment, the above-described sequence data 920 of the oligonucleotide may be interpreted as a concept including the plurality of the oligonucleotide sequence sets above-described.

As described above, the prediction result for the dimerization of the oligonucleotide may be obtained, using the method for predicting a dimerization in a nucleic acid amplification reaction described herein. Depending on the implementation, a technical feature for predicting a dimerization in the nucleic acid amplification reaction using the dimer prediction model may be used independently, without combining the technical feature with a technical feature for obtaining the dimer prediction model by using the transfer learning method.

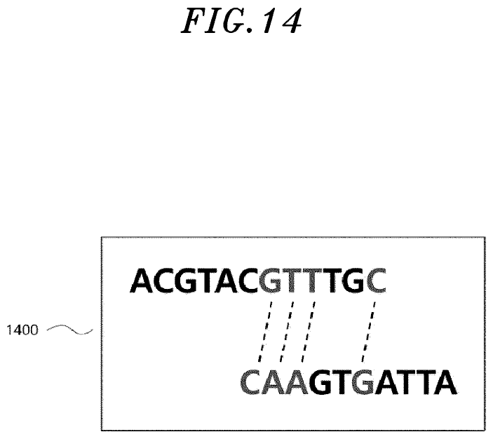

FIG. 14 is a view illustrating an example process in which the computer device 100 provides a predicted image representing the dimerization according to an embodiment.

Referring to FIG. 14, the computer device 100 may provide a predicted image 1400 representing the dimerization. In an embodiment, the computer device 100 may generate the predicted image 1400 for a predicted dimer binding between a forward primer sequence and a reverse primer sequence whose dimerization probability value 930 is not less than a preset reference value.

In an embodiment, the computer device 100 may generate the predicted image 1400 between the forward primer sequence and the reverse primer sequence based on the prediction supporting data described above. For example, the computer device 100 may derive positions of base pairs where dimer binding is predicted between the forward primer sequence and the reverse primer sequence, by using explainable features and values extracted by the XAI method. Further, the computer device 100 may generate the predicted image 1400 in which a bond line of each of the base pairs formed between the forward primer sequence and the reverse primer sequence is displayed by connecting the derived positions.

As an example, the computer device 100 may generate an annotation data as to the dimerization probability value 930 for each position of the bases included in the sequence of the oligonucleotide based on the prediction supporting data. The computer device 100 may add an identifiable indicator (e.g., highlight) to a base showing a higher contribution than a preset level to the dimerization probability value 930 among the bases shown in the predicted image 1400 using the generated annotation data.

In an embodiment, the computer device 100 may generate the predicted image 1400 by detecting a base pair of the forward primer sequence and the reverse primer sequence satisfying a plurality of pre-stored pattern rules corresponding to the dimerization. In an embodiment, the computer device 100 may indicate the prediction supporting data on the predicted image 1400 to provide them together.

The predicted image 1400 shown in FIG. 14 is used for illustrative purposes, and a type of image, a display method of factors corresponding to bases in an image, and a display method of the binding line between bases may vary depending on the implementation mode. In addition, depending on the implementation mode, a multi-dimensional (e.g., three-dimensional) molecular structure of the oligonucleotide, and a dimer structure representing a binding between molecules corresponding to the oligonucleotides, etc., in the predicted image 1400 may be expressed by using various types of data structures.

According to an embodiment of the present disclosure, the computer device 100 may provide a suitability and/or unsuitability determination result for the sequence data 920 of the oligonucleotide based on the prediction result for the dimerization described above. For example, if the dimerization probability value 930 predicted by the dimer prediction model 910 with respect to the sequence data 920 of the oligonucleotide is not larger than a preset first reference value, the computer device 100 may output the suitability determination result indicating that the sequence data 920 of the oligonucleotide is suitable for the target nucleic acid sequence. If the dimerization probability value 930 is not less than a preset second reference value, the computer device 100 may output the unsuitability determination result indicating that the sequence data 920 of the oligonucleotide is not suitable for the target nucleic acid sequence.

In an embodiment, the computer device 100 may obtain a first design list including a plurality of design candidate groups including a plurality of the sequence data 920 of the oligonucleotide. The computer device 100 may provide each input data including the sequence data 920 of the oligonucleotide in each design candidate group to the dimer prediction model 910, and obtain each prediction result for the dimerization corresponding to each design candidate group from the dimer prediction model 910. The computer device 100 may obtain a suitability and/or unsuitability determination result for each design candidate group by using each prediction result. The computer device 100 may provide a second design list in which the suitability and/or unsuitability determination result is added to the first design list, or one or more design candidate groups determined to be unsuitable is excluded from the first design list. Depending on the embodiment, the second design list may be used to design a diagnostic reagent (e.g., primer, probe) for detecting a preset specific target analyte.

Depending on the embodiment, since a possibility of a dimerization may be considered during a design process of oligonucleotide design, an oligonucleotide with the suitability determination result may have more robust performance. For example, when using an oligonucleotide sequence set with the suitability determination result according to an embodiment of the present disclosure, a possibility of false positive may be reduced.

Depending on the embodiment, since oligonucleotides with the unsuitability determination results may be excluded from diagnostic reagent candidates for detecting the target analyte, a use of oligonucleotides with a relatively low dimerization probability value 930 may be considered. As a result, oligonucleotides more specific to a particular organism may be designed.

In an embodiment, the computer device 100 may provide a warning message for one or more design candidate groups having the dimerization probability value 930 not less than the first reference value and not larger than the second reference value from the first design list. Alternatively, the computer device 100 may provide the warning message for P (P is a natural number) oligonucleotide sequence sets having the top P probability values when the dimerization probability values 930 of a plurality of oligonucleotide sequence sets are sorted in descending order.

In an embodiment, the computer device 100 may provide a recommendation message for Q (Q is a natural number) oligonucleotide sequence sets with the top Q probability values when the dimer formation probability values 930 of the plurality of oligonucleotide sequence sets are sorted in ascending order.

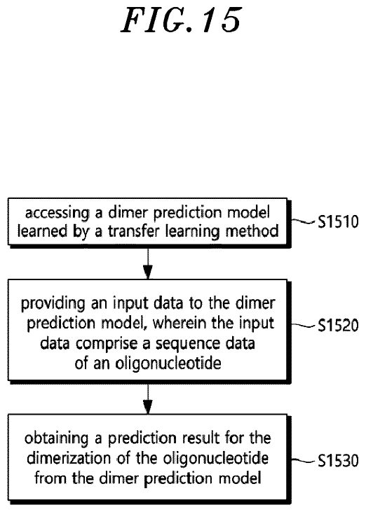

FIG. 15 is an exemplary flowchart for predicting a dimerization in a nucleic acid amplification reaction by the computer device according to an embodiment.

In an embodiment, steps shown in FIG. 15 may be performed by the computer device 100. In a further embodiment, the steps shown in FIG. 15 may be implemented by one entity, such as a method in which the steps are performed by the user terminal. In another further embodiment, the steps shown in FIG. 15 may be implemented by a plurality of entities, such as a method in which a part of the steps are performed by the user terminal and another part of the steps are performed by a server.

In Step S1510, the computer device 100 may access the dimer prediction model 910 learned by the transfer learning method.

In an embodiment, the computer device 100 may be implemented to access the dimer prediction model 910 by using a method, in which the user terminal receives the dimer prediction model 910 from the server and executes the dimer prediction model 910, wherein the dimer prediction model 910 is learned using the transfer learning method by the server. In an embodiment, the computer device 100 may be implemented to access the dimer prediction model 910 by using a method, in which the dimer prediction model 910 learned by the server is stored in a database and the user terminal receives the dimer prediction model 910 from the database and executes the dimer prediction model 910. In an embodiment, the computer device 100 may be implemented to access the dimer prediction model 910 by using a method, in which the user terminal loads the dimer prediction model 910 pre-stored in the memory 110 or another storage medium and executes the dimer prediction model 910. In an embodiment, the computer device 100 may be implemented to access the dimer prediction model 910 by using a method, in which the user terminal receives a result for the execution of the dimer prediction model 910 from the server by transmitting a request for execution of the dimer prediction model 910 learned by the server to the server along with a data required to execute the dimer prediction model 910 (e.g., input data, etc.). However, the accessing the dimer prediction model 910 in the disclosure is not limited to the embodiments disclosed herein, and various changes may be made thereto.

In another embodiment, the computer device 100 may be implemented to access the dimer prediction model 910 by loading a pre-stored dimer prediction model from the memory 110.

In Step S1520, the computer device 100 may provide an input data to the dimer prediction model 910, wherein the input data comprise the sequence data 920 of the oligonucleotide. In an embodiment, the computer device 100 may obtain the sequence data 920 of the oligonucleotide based on user input for a sequence of an oligonucleotide, or receive the sequence data 920 of the oligonucleotide from the memory 110, another device, or storage medium. In an embodiment, the oligonucleotide to be predicted may be one of diagnostic reagent candidates for detecting a specific target analyte. In an embodiment, the oligonucleotide may comprise a primer, for example, the oligonucleotide may comprise the forward primer and the reverse primer.

In the first embodiment, the dimer prediction model 910 may comprise the plurality of models generated by fine-tuning the pre-trained model 510 in accordance with each of reaction conditions used in the nucleic acid amplification reaction. The computer device 100 may input the sequence data 920 of the oligonucleotide into each of the plurality of models. Alternatively, based on a data of a reaction condition obtained together with the sequence data 920 of the oligonucleotide, the computer device 100 may select one or more models corresponding to the corresponding reaction condition from the plurality of models, and input the sequence data 920 of the oligonucleotide into each of the selected one or more models.

In the second embodiment, the dimer prediction model 910 may comprise one model generated by fine-tuning the pre-trained model 510 using the plurality of training data sets. Further, each training input data in each training data set may further comprise a data of the reaction condition used in the nucleic acid amplification reaction. The computer device 100 may generate the input data comprising the sequence data 920 of the oligonucleotide and the data of the reaction condition obtained together with the sequence data 920, and input the generated input data into the one model described above.

In Step S1530, the computer device 100 may obtain a prediction result for the dimerization of the oligonucleotide from the dimer prediction model 910.

In an embodiment, the prediction result for the dimerization may comprise the dimerization probability value 930. In an embodiment, the dimerization probability value 930 may be calculated in units of an oligonucleotide sequence set pairing the sequence 1011 of the first oligonucleotide and the sequence 1012 of the second oligonucleotide. The dimerization probability value 930 may comprise a quantitative value representing a probability of a dimerization for each sequence set. In another embodiment, the prediction result for the dimerization may comprise the classification result for the dimerization (e.g., ‘occurrence of dimer’ or ‘non-occurrence of dimer’). In another embodiment, the prediction result for the dimerization may comprise the prediction classification (e.g., ‘high level of dimerization probability’, ‘medium level of dimerization probability’, or ‘low level of dimerization probability’ determined by using the dimerization probability value 930.

In the first embodiment, the computer device 100 may obtain a plurality of prediction results from the plurality of models described above and output the prediction result for the dimerization comprising the plurality of prediction results and a description of the corresponding reaction conditions. Alternatively, the computer device 100 may obtain one or more prediction results from one or more models corresponding to reaction conditions matched to the input data among the plurality of models, and output the prediction result for the dimerization comprising that one or more obtained prediction results and a description of the corresponding reaction conditions.

In a second embodiment, the computer device 100 may obtain one prediction result obtained based on the sequence data 920 and the corresponding reaction condition from the above-described one model. Further, the computer device 100 may output the prediction result for the dimerization comprising that one obtained prediction result and a description of the corresponding reaction condition.

In an embodiment, the computer device 100 may output the prediction supporting data used as a basis for prediction of the dimerization. In an embodiment, the prediction supporting data may be calculated by the XAI method. In an embodiment, the computer device 100 may provide the predicted image 1400 representing the dimerization.

In an embodiment, the computer device 100 may provide a suitability and/or unsuitability determination result for the sequence data 920 of the oligonucleotide based on the prediction result for the dimerization described above. In an embodiment, the computer device 100 may obtain a first design list including a plurality of design candidate groups including a plurality of the sequence data 920 of the oligonucleotide. Further, the computer device 100 may obtain a suitability and/or unsuitability determination result for each design candidate group, by using each prediction result for the dimerization corresponding to each design candidate group obtained from the dimer prediction model 910. Further, the computer device 100 may provide a second design list in which one or more design candidate groups determined to be unsuitable is excluded from the first design list.

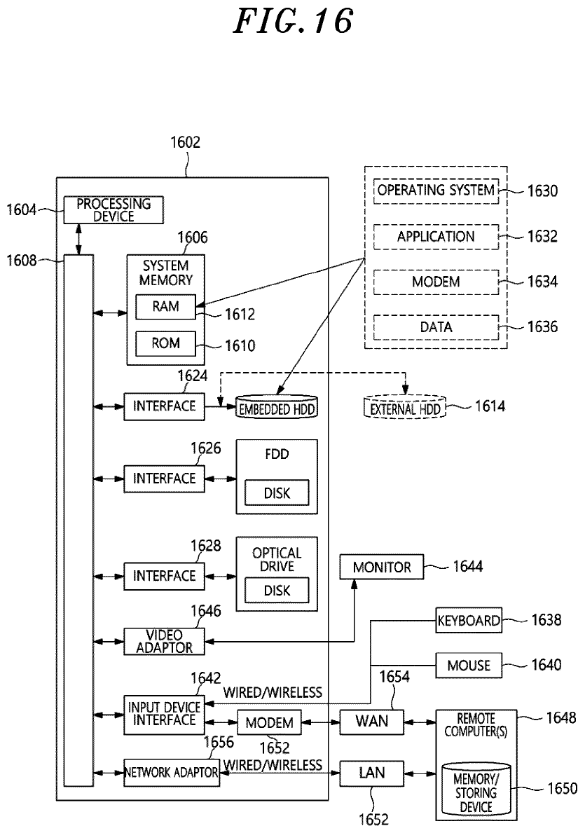

FIG. 16 is a schematic diagram illustrating a computing environment according to an exemplary embodiment of the present disclosure.

In the present disclosure, a component, a module, or a unit includes a routine, a procedure, a program, a component, a data structure, and the like for performing a specific task or implementing a specific abstract data type. Further, those skilled in the art will appreciate well that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The exemplary embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer device generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transmission medium.

The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

By way of example, not limitation, the computer 1602 in FIG. 16 may be interchangeably used with the computer device 100.

An illustrative environment 1600 including a computer 1602 and implementing several aspects of the present disclosure is illustrated, and the computer 1602 includes a processing device 1604, a system memory 1606, and a system bus 1608. The system bus 1608 connects system components including the system memory 1606 (not limited) to the processing device 1604. The processing device 1604 may be a predetermined processor among various commonly used processors. A dual processor and other multi-processor architectures may also be used as the processing device 1604.

The system bus 1608 may be a predetermined one among several types of bus structure, which may be additionally connectable to a local bus using a predetermined one among a memory bus, a peripheral device bus, and various common bus architectures. The system memory 1606 includes a ROM 1610, and a RAM 1612. A basic input/output system (BIOS) is stored in a non-volatile memory 1610, such as a ROM, an erasable and programmable ROM (EPROM), and an EEPROM, and the BIOS includes a basic routine helping a transport of information among the constituent elements within the computer 1602 at a time, such as starting. The RAM 1612 may also include a high-rate RAM, such as a static RAM, for caching data.

The computer 1602 also includes an embedded hard disk drive (HDD) 1614 (for example, enhanced integrated drive electronics (EIDE) and serial advanced technology attachment (SATA)), a magnetic floppy disk drive (FDD) 1616 (for example, which is for reading data from a portable diskette 1618 or recording data in the portable diskette 1618), and an SSD and an optical disk drive 1620 (for example, which is for reading a CD-ROM disk 1622, or reading data from other high-capacity optical media, such as a DVD, or recording data in the high-capacity optical media). A hard disk drive 1614, a magnetic disk drive 1616, and an optical disk drive 1620 may be connected to the system bus 1608 by a hard disk drive interface 1624, a magnetic disk drive interface 1626, and an optical drive interface 1628, respectively. An interface 1624 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1602, the drive and the medium correspond to the storage of random data in an appropriate digital form. In the description of the computer readable storage media, the HDD, the portable magnetic disk, and the portable optical media, such as a CD, or a DVD, are mentioned, but those skilled in the art will well appreciate that other types of computer readable storage media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1630, one or more application programs 1632, other program modules 1634, and program data 1636 may be stored in the drive and the RAM 1612. An entirety or a part of the operation system, the application, the module, and/or data may also be cached in the RAM 1612. It will be appreciated that the present disclosure may be implemented in a variety of commercially available operating systems or combinations of operating systems.

A user may input a command and information to the computer 1602 through one or more wired/wireless input devices, for example, a keyboard 1638 and a pointing device, such as a mouse 1640. Other input devices (not illustrated) may be a microphone, an IR remote controller, a joystick, a game pad, a stylus pen, a touch screen, and the like. The foregoing and other input devices are frequently connected to the processing device 1604 through an input device interface 1642 connected to the system bus 1608, but may be connected by other interfaces, such as a parallel port, an IEEE 1394 serial port, a game port, a USB port, an IR interface, and other interfaces.

A monitor 1644 or other types of display devices are also connected to the system bus 1608 through an interface, such as a video adaptor 1646. In addition to the monitor 1644, the computer generally includes other peripheral output devices (not illustrated), such as a speaker and a printer.

The computer 1602 may be operated in a networked environment by using a logical connection to one or more remote computers, such as remote computer(s) 1648, through wired and/or wireless communication. The remote computer(s) 1648 may be a workstation, a server computer, a router, a personal computer, a portable computer, a microprocessor-based entertainment device, a peer device, and other general network nodes, and generally includes some or an entirety of the constituent elements described for the computer 1602, but only a memory storage device 1650 is illustrated for simplicity. The illustrated logical connection includes a wired/wireless connection to a local area network (LAN) 1652 and/or a larger network, for example, a wide area network (WAN) 1654. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1602 is used in the LAN networking environment, the computer 1602 is connected to the local network 1652 through a wired and/or wireless communication network interface or an adaptor 1656. The adaptor 1656 may make wired or wireless communication to the LAN 1652 easy, and the LAN 1652 also includes a wireless access point installed therein for the communication with the wireless adaptor 1656. When the computer 1602 is used in the WAN networking environment, the computer 1602 may include a modem 1658, is connected to a communication server on a WAN 1654, or includes other means setting communication through the WAN 1654 via the Internet. The modem 1658, which may be an embedded or outer-mounted and wired or wireless device, is connected to the system bus 1608 through a serial port interface 1642. In the networked environment, the program modules described for the computer 1602 or some of the program modules may be stored in a remote memory/storage device 1650. The illustrated network connection is illustrative, and those skilled in the art will appreciate well that other means setting a communication link between the computers may be used.

The computer 1602 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

Meanwhile, disclosed is a computer readable medium storing the data structure according to an exemplary embodiment of the present disclosure.

The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device can perform operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO-Last in First Out) in which the data is input last and output first. The queue is a data listing structure that may access data limitedly and unlike a stack, the queue may be a data structure (FIFO-First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for training the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for training the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include training data input in a neural network training process and/or input data input to a neural network in which training is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example, and the present disclosure is not limited thereto.

The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable, and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example, and the present disclosure is not limited thereto.

As a non-limiting example, the weight may include a weight which varies in the neural network training process and/or a weight in which neural network training is completed. The weight which varies in the neural network training process may include a weight at a time when a training cycle starts and/or a weight that varies during the training cycle. The weight in which the neural network training is completed may include a weight in which the training cycle is completed. Accordingly, the data structure including the weight of the neural network may include a data structure including the weight which varies in the neural network training process and/or the weight in which neural network training is completed. Accordingly, the above-described weight and/or a combination of each weight are included in a data structure including a weight of a neural network. The data structure is just an example, and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, Trie, m-way search tree, AVL tree, and Red-Black Tree in a nonlinear data structure) to increase the efficiency of operation while using resources of the computing device to a minimum. The above-described matter is just an example, and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of training cycle iterations, weight initialization (for example, setting a range of weight values to be subjected to weight initialization), and Hidden Unit number (e.g., the number of hidden layers and the number of nodes in the hidden layer). The data structure is just an example, and the present disclosure is not limited thereto.

Those skilled in the art will appreciate that the various illustrative logical blocks, modules, processors, means, circuits, and algorithm operations described in relationship to the exemplary embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various exemplary embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

What is claimed is:

1. A computer-implemented method for predicting a dimerization in a nucleic acid amplification reaction, comprising:
accessing a dimer prediction model learned by a transfer learning method;
providing an input data to the dimer prediction model, wherein the input data comprise a sequence data of an oligonucleotide; and
obtaining a prediction result for the dimerization of the oligonucleotide from the dimer prediction model.

2. The computer-implemented method of claim 1, wherein the oligonucleotide comprises a primer.

3. The computer-implemented method of claim 2, wherein the oligonucleotide comprises a forward primer and a reverse primer.

4. The computer-implemented method of claim 1, wherein the dimerization comprises at least one selected from the group consisting of (i) a dimerization formed between two or more oligonucleotides and (ii) a dimerization formed in one oligonucleotide.

5. The computer-implemented method of claim 1, wherein the dimer prediction model is a model obtained by fine-tuning a pre-trained model.

6. The computer-implemented method of claim 5, wherein the pre-trained model uses a plurality of nucleic acid sequences as a training data.

7. The computer-implemented method of claim 6, wherein the plurality of nucleic acid sequences are obtained from a specific group of an organism.

8. The computer-implemented method of claim 6, wherein the pre-trained model is trained by a semi-supervised learning method performed in which a mask to some of bases in the nucleic acid sequences is applied and then an answer of the masked base is found.

9. The computer-implemented method of claim 8, wherein the pre-trained model is trained by using nucleic acid sequences tokenized with tokens each having two or more bases.

10. The computer-implemented method of claim 9, wherein the tokens comprise each bases tokenized by (i) dividing the nucleic acid sequences by k unit (wherein k is a natural number) or (ii) dividing the nucleic acid sequences by a function unit.

11. The computer-implemented method of claim 5, wherein the fine-tuning is performed using a plurality of training data sets, each training data set comprises (i) a training input data comprising a sequence data of two or more oligonucleotides and (ii) a training answer data comprising a label data as to occurrence and/or non-occurrence of dimer of the two or more oligonucleotides.

12. The computer-implemented method of claim 11, wherein the fine-tuning comprises (i) joining sequences of the two or more oligonucleotides by using a discrimination token and (ii) tokenizing the joined sequences to obtain a plurality of tokens.

13. The computer-implemented method of claim 12, wherein the fine-tuning comprises (i) predicting a dimerization of the two or more oligonucleotides using a context vector generated from the plurality of tokens, and (ii) training the pre-trained model for reducing a difference between the predicted result and the training answer data.

14. The computer-implemented method of claim 5, wherein the dimer prediction model comprises a plurality of models generated by fine-tuning the pre-trained model in accordance with each of reaction conditions used in the nucleic acid amplification reaction.

15. The computer-implemented method of claim 14, wherein obtaining the prediction result comprises obtaining a plurality of prediction results for the dimerization from the plurality of models or obtaining a prediction result from a model corresponding to a reaction condition matched to the input data among the plurality of models.

16. The computer-implemented method of claim 11, wherein the training input data further comprises a data of a reaction condition used in the nucleic acid amplification reaction, and the dimer prediction model comprises one model generated by fine-tuning the pre-trained model using the plurality of training data sets.

17. The computer-implemented method of claim 16, wherein the input data further comprises a data of a reaction condition, whereby the prediction result for the dimerization is obtained based on the sequence data and the data of the reaction condition.

18. The computer-implemented method of claim 14, wherein the reaction condition is a reaction medium, a reaction temperature and/or a reaction time used in the nucleic acid amplification reaction.

19. A computer-readable recording medium storing a computer program including instructions that, when executed by one or more processors, enable the one or more processors to perform a method for predicting a dimerization in a nucleic acid amplification reaction by a computer device,
the method comprising:
accessing a dimer prediction model learned by a transfer learning method;
providing an input data to the dimer prediction model, wherein the input data comprise a sequence data of an oligonucleotide; and
obtaining a prediction result for the dimerization of the oligonucleotide from the dimer prediction model.

20. A computer device for predicting a dimerization in a nucleic acid amplification reaction, the computer device comprising:
a processor; and
a memory that stores one or more instructions that, when executed by the processor, cause the computer device to perform operations, the operations comprising:
accessing a dimer prediction model learned by a transfer learning method;
providing an input data to the dimer prediction model, wherein the input data comprise a sequence data of an oligonucleotide; and
obtaining a prediction result for the dimerization of the oligonucleotide from the dimer prediction model.
